(19) United States
(12) Patent Application Publication
(10) Pub. No.: US 2023/0095530 A1
(43) Pub. Date: Mar. 30, 2023

(54) COMPOUND USED AS RET KINASE INHIBITOR AND APPLICATION THEREOF

(71) Applicant: TYK Medicines, Inc., Huzhou (CN)

(72) Inventors: Yusheng Wu, Huzhou (CN); Jun Li, Huzhou (CN); Maolin Zheng, Huzhou (CN); Chengshan Niu, Huzhou (CN); Apeng Liang, HuZhou (CN)

(73) Assignee: TYK Medicines, Inc, Huzhou (CN)

(21) Appl. No.: 17/789,115

(22) PCT Filed: Dec. 25, 2020
(86) PCT No.: PCT/CN2020/139655

§ 371 Date: Jun. 24, 2022

(30) Foreign Application Priority Data
Dec. 27, 2019 (CN) 201911380371.6

(51) Int. Cl. A61K 31/4995; C07D 471/04; C07D 487/08; A61K 31/444; A61K 31/4545; A61K 39/395; A61K 31/675; A61K 31/4745; A61K 31/553; A61K 31/5377; A61K 31/519; A61K 31/4985; A61K 31/44; A61K 31/517; A61K 31/325

(52) U.S. Cl. CPC A61K 31/4995; C07D 471/04; C07D 487/08; A61K 31/444; A61K 31/4545; A61K 39/3955; A61K 31/675; A61K 31/4745; A61K 31/553; A61K 31/5377; A61K 31/519; A61K 31/4985; A61K 31/44; A61K 31/517; A61K 31/325

(57) ABSTRACT

The present invention relates to a compound used as an RET kinase inhibitor and an application thereof. The compound has a structure represented by formula F, has a good inhibitory ability for RET kinase, and has relatively good pharmacodynamic and pharmacokinetic performance, and lower toxic side effects.

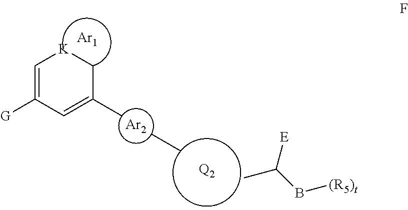

### TECHNICAL FIELD

The invention relates to the technical field of medicine, in particular to a compound used as RET kinase inhibitor and application thereof in regulating RET kinase activity or treating RET-related diseases.

### BACKGROUND OF THE INVENTION

RET (Rearranged during transfection) gene is located on chromosome 10. RET protein encoded thereby is a receptor tyrosine kinase (RTK) which exists on cell membrane. Its mutation types mainly include fusion mutation with KIF5B, TRIM33, CCDC6 and NCOA4 genes, and point mutation such as M918T. RET is a receptor tyrosine kinase, which is related to the signal transduction of cell proliferation, migration and differentiation, the survival of neural crest cells, the formation of kidney organs, spermatogenesis and other processes. Its abnormal expression, mutation and recombination are closely related to the occurrence and development of many cancers, such as mastoid thyroid carcinoma, multiple endocrine adenomatosis type 2, medullary thyroid carcinoma, pheochromocytoma and paratyroid adenoma, etc. In lung cancer, abnormal recombination of RET gene KIF5B-RET and CCDC6-RET is related to about 1-2% of lung adenocarcinoma, of which KIF5B-RET accounts for 70-90% and CCDC6-RET accounts for about 10-25%. At present, the treatment scheme for RET gene change is mainly to use multi-kinase inhibitors, such as carbotinib and vandetanil. Because of low targeting, serious toxicity associated with VEGFR inhibition due to off-target usually occurs.

Blueprint and Loxo Oncology have disclosed their highly effective and selective oral RET inhibitors BLU-667 and LOXO-292. The results of Blueprint phase I clinical data show that BLU-667 shows broad anti-tumor activity, and the overall remission rate (ORR) in tumor patients with RET fusion and mutation is 45%, among which the ORR in patients with non-small cell lung cancer and medullary thyroid carcinoma is 50% and 40%, respectively. Recently, FDA granted LOXO-292, a research drug of Loxo Oncology Company, as a breakthrough therapy for treating patients with non-small cell lung cancer (NSCLC) and medullary thyroid cancer (MTC) carrying RET genetic variation.

Both BLU-667 and LOXO-292 are still in clinical trial stage. Therefore, developing novel compounds with RET kinase inhibitory activity and better pharmacodynamic and pharmacokinetic properties has become an important research project to develop new anti-tumor drugs which can be finally used in the treatment of human tumors and other diseases.

### SUMMARY OF THE INVENTION

The object of the present invention is to provide a novel class of compounds with RET kinase inhibitory activity and/or better pharmacodynamics/pharmacokinetic properties and use thereof.

In the first aspect of the invention, it provides a compound of formula F. or a pharmaceutically acceptable salt, stereoisomer, solvate or prodrug thereof,

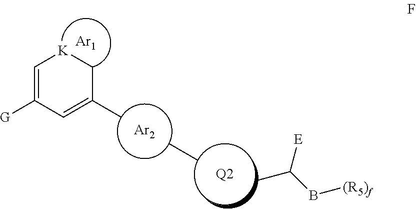

wherein,

G is selected from A-Z₁— or D:

Ar₁ is a substituted or unsubstituted 5-6-membered heteroaryl containing 1-4 N atoms, wherein the “substituted” means being substituted by one or more groups selected from the group consisting of H. CN, halogen, methyl, ethyl and cyclopropyl;

Ar₂ is selected from the substituted or unsubstituted group consisting of 5-6-membered aryl and 5-6-membered heteroaryl, wherein the “substituted” means being substituted by one or more groups selected from the group consisting of C1-C6 alkyl, halogen, hydroxyl, oxo (═O), C1-C6 heteroalkyl, C1-C6 alkoxy, C3-C14 cycloalkyl, 3-14-membered heterocycloalkyl and cyano;

K is selected from C or N:

Q₂ is selected from the group consisting of saturated 4-7-membered monocyclic heterocyclyl, saturated 7-8-membered bridged heterocyclyl, saturated 7-11-membered spiro heterocyclyl,

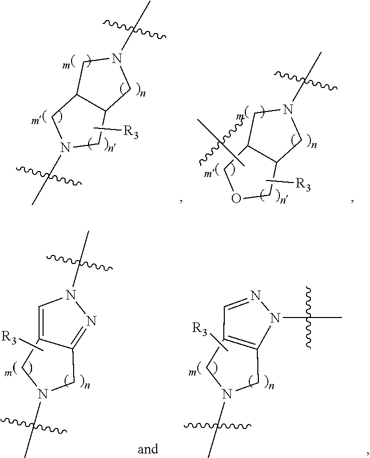

wherein the heterocyclyl contains 1, 2 or 3 nitrogen heteroatoms as a ring skeleton, and m, n, m′ and n′ are each independently 0, 1, 2 or 3;

and the H on Q₂ may optionally be substituted by one or more substituents selected from the group consisting of deuterium, hydroxy, halogen, cyano, ester, amido, carbonyl, oxo (═O), amino, C1-C6 alkyl, C1-C6 haloalkyl, C1-C6 thioalkyl, C1-C6 alkoxy, C1-C6 heteroalkyl, C1-C6 alkylamino, C3-C6 cycloalkyl, C3-C8 cycloalkylamino, C6-C14 aryl and 5-14-membered heteroaryl; R₃ is a substituted or unsubstituted 5-6-membered heteroaryl, a C1-C6 alkyl or a C1-C6 heteroalkyl, which may optionally be substituted by one or more C1-C6 alkyl;

B is independently selected from the substituted or unsubstituted group consisting of 3-7-membered ring, C6-C14 aryl, 5-14-membered heteroaryl, 7-20-membered spiro or bridged ring, and the ring contains 0-3 heteroatoms selected from N, O or S; the “substituted” means being substituted by one or more substituents selected from the group consisting of deuterium, hydroxy, halogen, cyano, ester, amido, carbonyl, amino, C1-C6 alkyl, C1-C6 haloalkyl, C1-C6 thioalkyl, C1-C6 alkoxy, C1-C6 heteroalkyl, C1-C6 alkylamino, C3-C6 cycloalkyl, C3-C8 cycloalkylamino, C6-C14 aryl and 5-14-membered heteroaryl;

E is independently selected from the substituted or unsubstituted group consisting of hydrogen, C1-C6 alkyl, C1-C6 alkoxy, C3-C6 cycloalkyl, C1-C6 heteroalkyl, and 3-6-membered heterocyclyl, wherein the “substituted” means being substituted by 0-5 R^{a}:

each R₅ is independently selected from the substituted or unsubstituted group consisting of hydrogen, nitro, cyano, halogen, C1-C6 alkyl, C2-C6 alkenyl, C2-C6 alkynyl, C1-C6 alkoxy, C1-C6 heteroalkyl, C3-C12 cycloalkyl, C6-C14 aryl, 5-14-membered heteroaryl, C6-C14 aryloxy, C6-C14 aryl C1-C6 alkyl, 3-12-membered heterocyclyl, 3-12-membered heterocycloalkyl, —C(O)R₆, —OC(O)R₆, —C(O)OR₆, —(C1-C6 alkylene)-C(O)R₆, —SR₆, —S(O)₂R₆, —S(O)₂—N(R₆)(R₇), —(C1-C6 alkylene)-S(O)₂R₆, —(C1-C6 alkylene)-S(O)₂—N(R₆)(R₇), —N(R₆)(R₇), —C(O)—N(R₆)(R₇), —N(R₆)—C(O)R₇, —N(R₆)—C(O)OR₇—, —(C1-C6 alkylene)-N(R₆)—C(O)R₆, —N(R₆)S(O)₂R— and —P(O)(R₆)(R₇); wherein, the “substituted” means being substituted by 0, 1, 2, 3, 4 or 5 R^{a}; R₆ and R₇ are each independently selected from the group consisting of C1-C6 alkyl, C2-C6 alkenyl, C2-C6 alkynyl, C1-C6 alkoxy, C1-C6 heteroalkyl, C3-C6 cycloalkyl, C6-C14 aryl, 5-14-membered heteroaryl, C6-C14 aryloxy, C6-C14 aryl C1-C6 alkyl, C3-C6 heterocycloalkyl, C1-C6 alkylamino, and C3-C6 cycloalkylamino; or R₆ and R₇ together with their adjacent N atom form a substituted or unsubstituted 3-6-membered heterocyclyl; wherein the “substituted” means being substituted by 0, 1, 2, 3, 4 or 5 R^{a};

A is independently selected from the group consisting of H, substituted or unsubstituted C1-C6 alkyl, substituted or unsubstituted 4-6-membered heterocyclyl, and (R₁R₂N) C(═O)—; wherein the “substituted” means being substituted by one or more groups selected from the group consisting of halogen. —OH, C1-C6 alkoxy, C1-C6 alkyl, amino, 5-6-membered heteroaryl, 4-6-membered heterocyclyl, C3-C6 cycloalkyl, amido, (R₁R₂N) C(═O)—, hydroxyC1-C6 alkyl, (C1-C6 alkyl) C(═O)—, C1-C6 alkoxy, oxo and (C1-C6 alkoxy) C(═O)—; R₁ and R₂ are each independently selected from H or C1-C6 alkyl, wherein the alkyl may optionally be substituted by 1-3 fluorine;

Z₁ is selected from the group consisting of NR^{b}, S—, —C(R^{b}R^{c})— and —O—;

D is a 5-14-membered heteroaryl, wherein H on the heteroaryl is optionally substituted by one or more substituents selected from the group consisting of deuterium, hydroxy, halogen, cyano, ester, amido, oxo, amino, C1-C6 alkyl, C1-C6 alkoxy, C1-C6 heteroalkyl, C3-C6 cycloalkyl, C3-C8 cycloalkylamino, C6-C14 aryl and 5-14-membered heteroaryl; the C1-C6 alkyl, C1-C6 alkoxy, C1-C6 heteroalkyl, C3-C6 cycloalkyl, C3-C8 cycloalkylamino, C6-C14 aryl or 5-14-membered heteroaryl may be further substituted by one or more groups selected from the group consisting of halogen, cyano and hydroxyl;

f is 0, 1, 2, 3, 4, 5 or 6;

R^{a} is independently selected from the group consisting of O, C1-C6 alkyl, halogen, hydroxyl, C1-C6 heteroalkyl, C1-C6 alkoxy, C3-C14 cycloalkyl, 3-14-membered heterocycloalkyl and cyano;

R^{b} and R^{c} are independently selected from the group consisting of H, C1-C6 alkyl, halogen, hydroxyl, C1-C6 heteroalkyl, C1-C6 alkoxy, C3-C14 cycloalkyl, 3-14-membered heterocycloalkyl and cyano;

with the proviso that

when

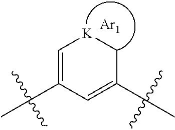

is

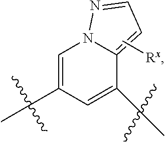

Ar₂ is a 5-6-membered heteroaryl, and Ar₂ connects with ring Q₂ and/or

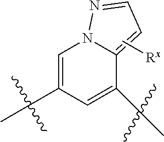

through N; wherein R^{x} is selected from the group consisting of H, CN, halogen, methyl, ethyl and cyclopropyl; the H atom on Ar₂ can be substituted by CR^{a}.

In another preferred embodiment,

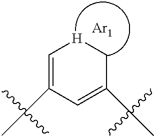

is selected from

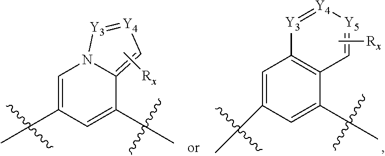

wherein, Rₓ is selected from the group consisting of H, CN, halogen, methyl, ethyl and cyclopropyl; Y₃, Y₄ and Y₅ are N or CR^{a}, R^{a} is defined as above.

In another preferred embodiment,

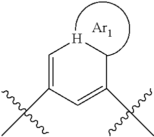

is selected from

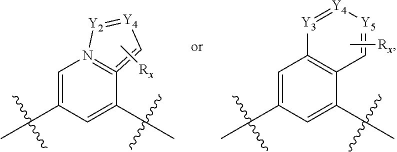

wherein, Rₓ is selected from the group consisting of H, CN, halogen, methyl, ethyl and cyclopropyl; Y₃, Y₄ and Y₅ are CH, N or CR^{a}, R^{a} is defined as above.

In another preferred embodiment, Ar₂ is selected from

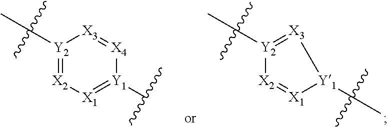

wherein

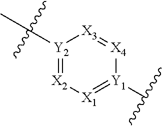

is a six-membered heteroaryl;

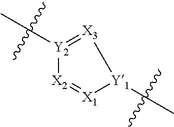

is a five-membered heteroaryl; X₁, X₂, X₃ and X₄ are each independently CH, N or CR^{a}, and 0, 1 or 2 of X₁, X₂, X₃ and X₄ are N; Y′₁ is N; Y₁ is C or N; Y₂ is N or C.

In another preferred embodiment, Ar₂ is selected from

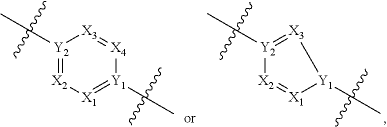

wherein, Y₁ and Y₂ are CR^{a} or N, X₁, X₂, X₃ and X₄ are each independently selected from CR^{a} or N, and 0, 1 or 2 of X₁, X₂, X₃ and X₄ are N; R^{a} is independently selected from the group consisting of O, C1-C6 alkyl, halogen, hydroxyl, C1-C6 heteroalkyl, C1-C6 alkoxy, C3-C14 cycloalkyl, 3-14-membered heterocycloalkyl and cyano.

In another preferred embodiment, Ar₂ is selected from

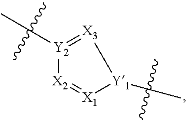

wherein, Y′₁ is N, Y₂ is CR^{a} or N; X₁, X₂ and X₃ are each independently selected from CH, CR^{a} or N, and 0, 1 or 2 of X₁, X₂ and X₃ are N; R^{a} is independently selected from the group consisting of O, C1-C6 alkyl, halogen, hydroxyl, C1-C6 heteroalkyl, C1-C6 alkoxy, C3-C14 cycloalkyl, 3-14-membered heterocycloalkyl and cyano.

In another preferred embodiment, the compound, or the pharmaceutically acceptable salt, stereoisomer, solvate or prodrug thereof has the structure represented by formula (F-I), formula (F-II), or formula (F-III),

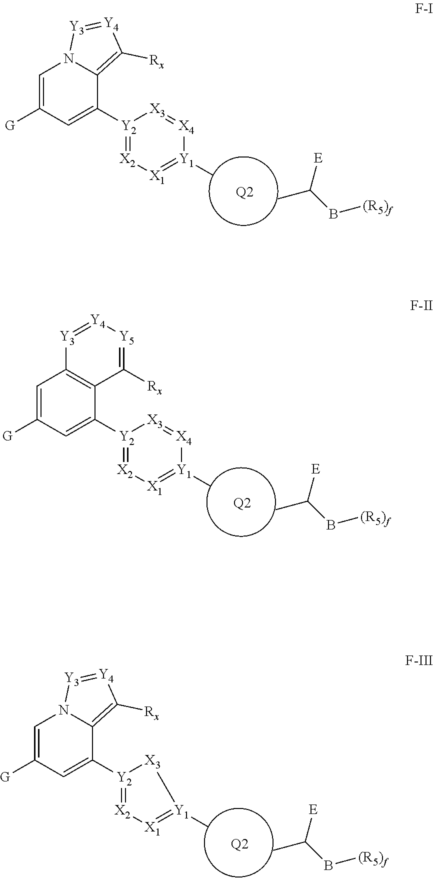

X₁, X₂, X₃ and X₄ are each independently selected from N or CR^{a}, and 0, 1 or 2 of X₁, X₂, X₃, and X₄ are N; Y₁, Y₃ and Y₅ are each independently selected from N or CR^{a}, Y₂ and Y₄ are each independently N or C;

Rₓ is independently selected from the group consisting of H, CN, halogen, methyl, ethyl and cyclopropyl:

G, Q₂, E, B, R₅, f, R^{a} are defined as above;

with the proviso that in formula F-I, when Y₃ is N and Y₄ is C, Y₁ and/or Y₂ is N.

In another preferred embodiment, the compound, or the pharmaceutically acceptable salt, stereoisomer, solvate or prodrug thereof has the structure represented by formula (I), formula (II) formula (III) formula (IV), formula (V) or formula (VI),

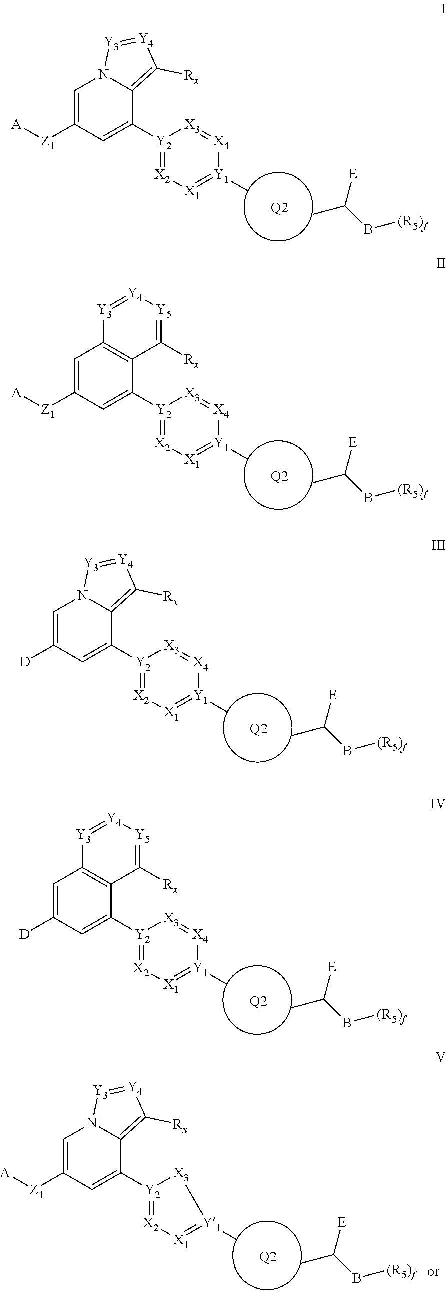

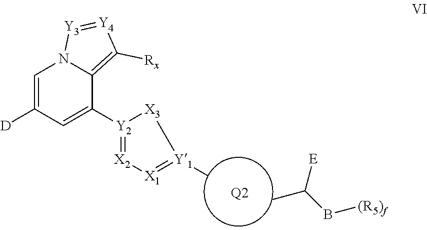

wherein,

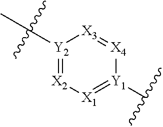

is a six-membered heteroaryl;

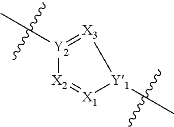

is a five-membered heteroaryl:

X₁, X₂, X₃ and X₄ are each independently CH, N or CR^{a}, and 0, 1 or 2 of X₁, X₂, X₃ and X₄ are N;

Y′₁ is N;

Y₁ is C or N;

Y₃ and Y₅ are each independently CH, N or CR^{a}; Y₂ is N or C;

Y₄ is CH, N, or CR^{a};

Rₓ is independently selected from the group consisting of H, CN, halogen, methyl, ethyl and cyclopropyl;

E, R₅, C, A, Z₁, D, Q₂, B and R^{a} are defined as above;

with the proviso that in formula I and formula III, when Y₃ is N and Y₄ is CH or N, Y₁ and/or Y₂ are N.

In another preferred embodiment, the compound, or the pharmaceutically acceptable salt stereoisomer, solvate or prodrug thereof, wherein Ar₁ is substituted or unsubstituted group consisting of

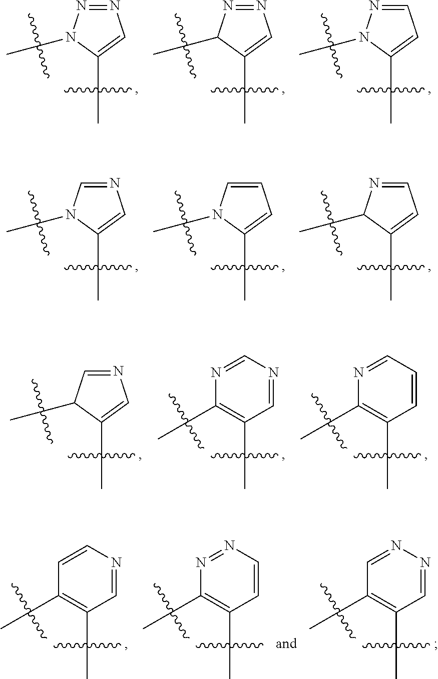

and wherein the “substituted” means being substituted by one or more groups selected from the group consisting of H, CN, halogen, methyl, ethyl and cyclopropyl.

In another preferred embodiment, A is independently selected from the group consisting of

a) H;

b) C1-C6 alkyl, optionally substituted by 1 to 3 groups selected from the group consisting of F, OH, R₃, R₄, C3-C6 cycloalkyl, (R₁R₂N) C(═O)— and R₁R₂N—; wherein the C3-C6 cycloalkyl may optionally be further substituted by OH;

c) R₄; or

d) (R₁R₂N) C(═O)—;

R₁ and R₂ are each independently selected from the group consisting of H and C1-C6 alkyl, wherein the alkyl may optionally be substituted by 1-3 fluorines; R₃ is a substituted or unsubstituted 5-6-membered heteroaryl having 1-3 ring heteroatoms selected from the group consisting of N, O and S, and the “substituted” means being substituted by one or more C1-C6 alkyl;

R₄ is a 4-6-membered heterocyclyl having 1-2 ring heteroatoms independently selected from N or O and optionally substituted by one or more substituents independently selected from the group consisting of OH, C1-C6 alkyl (optionally substituted by 1-3 fluorines), hydroxyl C1-C6 alkyl, halogen, (C1-C6 alkyl) C(═O)—, C1-C6 alkoxy, oxo and (C1-C6 alkoxy) C(═O)—.

In another preferred embodiment, A is C2-C6 alkyl, which may be optionally substituted by one or more groups selected from the group consisting of —OH, F and C3-C6 cycloalkyl.

In another preferred embodiment, A is —C1-C3 alkyl, which can optionally be substituted by one or more groups selected from the group consisting of C3-C6 cycloalkyl, C1-C6 alkoxy and R₄; wherein, C1-C6 alkoxy can be further substituted by 1˜3F; R₄ is a 4-6-membered heterocyclyl having 1-2 ring heteroatoms independently selected from N or O and optionally substituted by one or more substituents independently selected from the group consisting of OH, C1-C6 alkyl (optionally substituted by 1-3 fluorines), hydroxyl C1-C6 alkyl, halogen, (C1-C6 alkyl) C(═O)—, C1-C6 alkoxy, oxo and (C1-C6 alkoxy) C (═O)—.

In another preferred embodiment, A is a dihydroxvC3-C6 alkyl, which can optionally be substituted by C3-C6 cycloalkyl.

In another preferred embodiment, X₁ is N.

In another preferred embodiment, Z₁ is O.

In another preferred embodiment, Y₄ is N.

In another preferred embodiment, Y₅ is C.

In another preferred embodiment, X₁ is N, X₂, X₃ and X₄ are C.

In another preferred embodiment, E is hydrogen or substituted or unsubstituted C1-C6 alkyl, wherein the “substituted” means being substituted by 0-5 R^{a}, R^{a} is defined as above.

In another preferred embodiment, A is substituted or unsubstituted C2-C6 alkyl-OH, wherein the “substituted” means being substituted by one or more groups selected from the group consisting of fluorine and C3-C6 cycloalkyl.

In another preferred embodiment, Q₂ is a saturated 4-7-membered monocyclic heterocyclyl containing one or two nitrogen ring heteroatoms, and the H on Q₂ may optionally be substituted by one or more substituents selected from the group consisting of deuterium, hydroxy, halogen, cyano, ester, amido, carbonyl, oxo (═O), amino, C1-C6 alkyl, C1-C6 haloalkyl, C1-C6 thioalkyl, C1-C6 alkoxy, C1-C6 heteroalkyl, C1-C6 alkylamino, C3-C6 cycloalkyl, C3-C8 cycloalkylamino, C6-C14 aryl and 5-14-membered heteroaryl; preferably, the ring Q₂ is a 5-6 membered monocyclic nitrogen-containing heterocyclic ring.

In another preferred embodiment, Q₂ is a saturated 7-8-membered bridged heterocyclyl containing one or two nitrogen ring heteroatoms, and the H on Q₂ may optionally be substituted by one or more substituents selected from the group consisting of deuterium, hydroxy, halogen, cyano, ester, amido, carbonyl, oxo (═O), amino, C1-C6 alkyl, C1-C6 haloalkyl, C1-C6 thioalkyl, C1-C6 alkoxy, C1-C6 heteroalkyl, C1-C6 alkylamino, C3-C6 cycloalkyl, C3-C8 cycloalkylamino, C6-C14 aryl and 5-14-membered heteroaryl; preferably Q₂ is a 7-8-membered bridged nitrogen-containing heterocyclic ring.

In another preferred embodiment, Q₂ is a saturated 7-11-membered spiro heterocyclyl containing one or two nitrogen ring heteroatoms, and the H on Q₂ may optionally be substituted by one or more substituents selected from the group consisting of deuterium, hydroxy, halogen, cyano, ester, amido, carbonyl, oxo (═O), amino, C1-C6 alkyl, C1-C6 haloalkyl, C1-C6 thioalkyl, C1-C6 alkoxy, C1-C6 heteroalkyl, C1-C6 alkylamino, C3-C6 cycloalkyl, C3-C8 cycloalkylamino, C6-C14 aryl and 5-14-membered heteroaryl.

In another preferred embodiment, Q₂ is

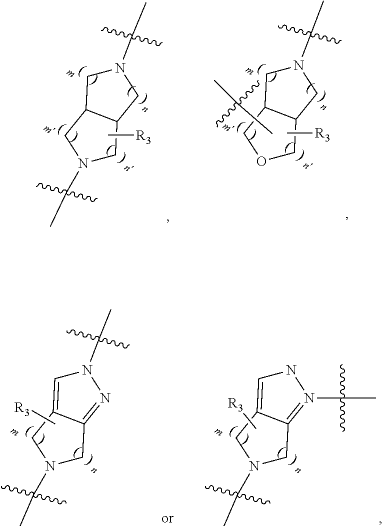

wherein m, n, m′ and n′ are each independently 0, 1, 2 or 3, R₃ is defined as above.

In another preferred embodiment, X₁ is N.

In another preferred embodiment, Q₂ is a saturated 5-6-membered monocyclic heterocyclyl containing one or two nitrogen ring heteroatoms, and the H on Q₂ may optionally be substituted by one or more substituents selected from the group consisting of deuterium, hydroxy, halogen, cyano, ester, amido, oxo, carbonyl, amino, C1-C6 alkyl, C1-C6 haloalkyl, C1-C6 thioalkyl, C1-C6 alkoxy, C1-C6 heteroalkyl, C1-C6 alkylamino, C3-C6 cycloalkyl, C3-C8 cycloalkylamino, C6-C14 aryl and 5-14-membered heteroaryl.

In another preferred embodiment, Y₃ is N.

In another preferred embodiment, B is a 5-6-membered heteroaryl and the H on B may optionally be substituted by one or more substituents selected from the group consisting of deuterium, hydroxy, halogen, cyano, ester, amido, carbonyl, amino, C1-C6 alkyl, C1-C6 haloalkyl, C1-C6 thioalkyl, C1-C6 alkoxy, C1-C6 heteroalkyl, C1-C6 alkylamino, C3-C6 cycloalkyl, C3-C8 cycloalkylamino, C6-C14 aryl and 5-14-membered heteroaryl.

In another preferred embodiment, the 5- or 6-membered heteroaryl in Ar₂ is

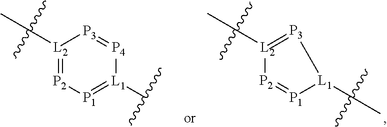

wherein, P₁, P₂, P₃ and P₄ are each independently selected from N or CH, wherein, 0, 1 or 2 of P₁, P₂, P₃ and P₄ are N, L₁ and L₂ are each independently selected from N or C.

In another preferred embodiment. Ar₂ is selected from

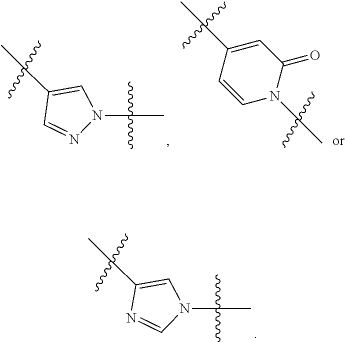 [SMILES: *c1cnn(*)c1 ; *c1ccn(*)c(=O)c1 ; *c1cn(*)cn1]

In another preferred embodiment, the compound, or the pharmaceutically acceptable salt, stereoisomer, solvate or prodrug thereof has the structure represented by formula (VII), formula (VIII) or formula (IX),

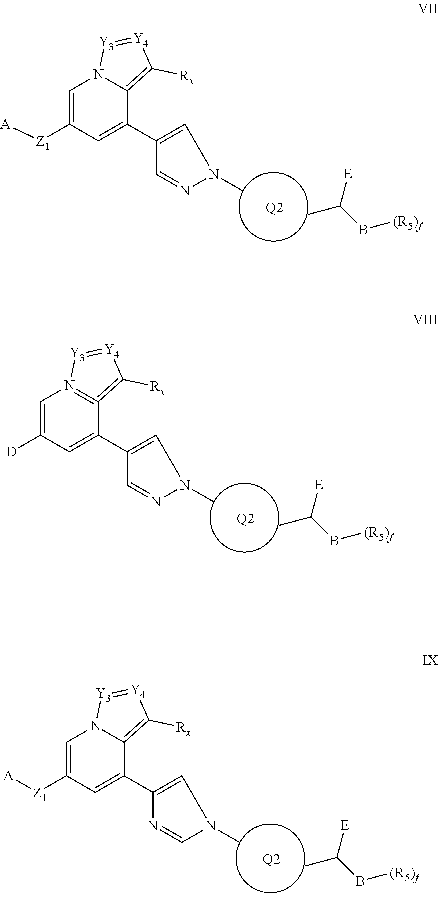

wherein, A, Z₁, D, Rₓ, Q₂, E, B, R₅ and f are defined as above; Y₃ and Y₄ are each independently CH, N or CR^{a}.

In another preferred embodiment, the compound, or the pharmaceutically acceptable salt, stereoisomer, solvate or prodrug thereof has the structure represented by formula (XI), or formula (XIII),

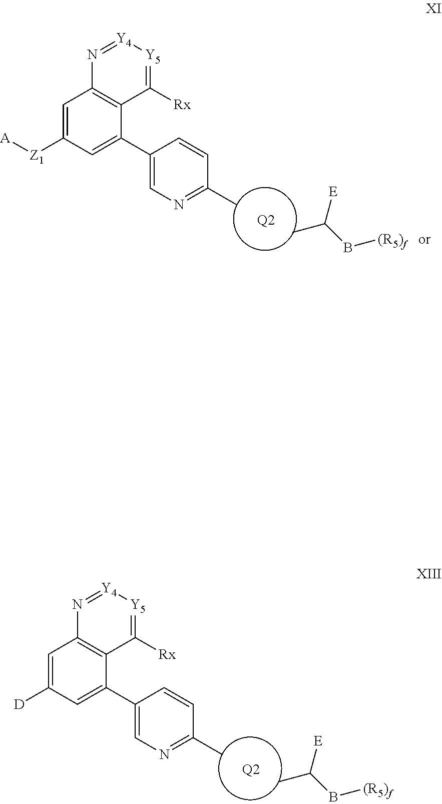

wherein Y₄ and Y₅ are each independently CH, N, or CR^{a};

A, Z₁, D, Rₓ, Q₂, E, B, R₅ and f are defined as above.

In another preferred embodiment, the B is selected from the substituted or unsubstituted group consisting of C6-C10 aryl and 5-10-membered heteroaryl, wherein the “substituted” means being substituted by one or more substituents selected from the group consisting of deuterium, halogen, C1-C6 alkyl, C1-C6 alkoxy, C1-C6 alkylamino, C3-C6 cycloalkyl, C3-C8 cycloalkylamino, C6-C14 aryl and 5-14-membered heteroaryl.

In another preferred embodiment, the compound, or the pharmaceutically acceptable salt, stereoisomer, solvate or prodrug thereof has the structure represented by formula

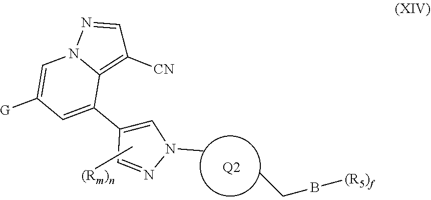

wherein,

each Rₘ is independently selected from C1-C6 alkyl, halogen, hydroxyl, oxo (═O), C1-C6 heteroalkyl, C1-C6 alkoxy, C3-C14 cycloalkyl, 3-14-membered heterocycloalkyl or cyano;

h is 0, 1 or 2:

G, B, Q₂, R₅ and f are defined as above.

In another preferred embodiment, Q2 is

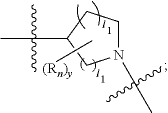

wherein, l₁ and l₂ are each independently 0, 1, 2 or 3, and l₁+l₂ is an integer of 1-4:

y is 0, 1, 2 or 3;

Rn is selected from deuterium, hydroxy, halogen, cyano, ester, amido, carbonyl, oxo (═O), amino, C1-C6 alkyl, C1-C6 haloalkyl. C1-C6 thioalkyl, C1-C6 alkoxy, C1-C6 heteroalkyl, C1-C6 alkylamino, C3-C6 cycloalkyl, C3-C8 cycloalkylamino, C6-C14 aryl or 5-14-membered heteroaryl.

In another preferred embodiment, G is selected from

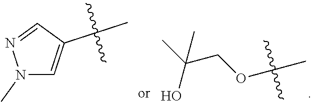

In another preferred embodiment, R₅ is selected from C1-C3 alkoxy or

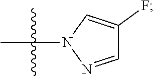 [SMILES: *n1cc(F)cn1]

preferably, R₅ is selected from methoxy or

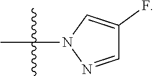 [SMILES: *n1cc(F)cn1]

In another preferred embodiment, Q₂ is selected from

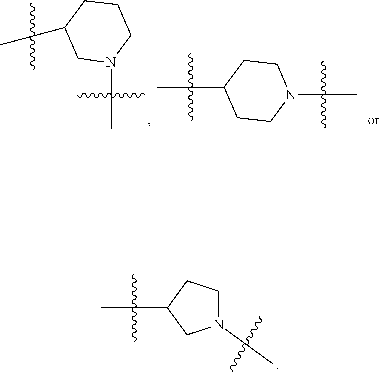 [SMILES: *C1CCCN(*)C1 ; *C1CCN(*)CC1 ; *C1CCN(*)C1]

In another preferred embodiment, B is substituted or unsubstituted group consisting of pyridyl, pyrimidinyl and thiazolyl; wherein the “substituted” means being substituted by one or more substituents selected from the group consisting of deuterium, hydroxy, halogen, cyano, ester, amido, carbonyl, amino, C1-C6 alkyl, C1-C6 haloalkyl, C1-C6 thioalkyl, C1-C6 alkoxy, C1-C6 heteroalkyl, C1-C6 alkylamino, C3-C6 cycloalkyl, C3-C8 cycloalkylamino, C6-C14 aryl and 5-14-membered heteroaryl.

In another preferred embodiment,

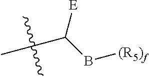

moiety is selected from

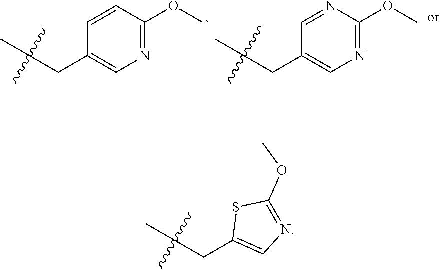

In another preferred embodiment, the compound, or the pharmaceutically acceptable salt, stereoisomer, solvate or prodrug thereof, does not contain

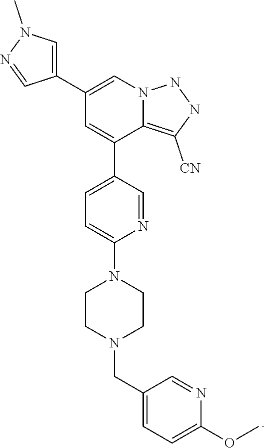 [SMILES: COc1ccc(CN2CCN(c3ccc(C4=CC(c5cnn(C)c5)=CN5NNC(C#N)=C45)cn3)CC2)cn1]

In another preferred embodiment, G, K, Ar₁, Ar₂, Q₂, E, B, R₅, f, Rₓ, A, Z₁, X₁, X₂, X₃, X₄, Y₁, Y₂, Y₃, Y₄ and Y₅ are the specific group corresponding to each specific compound in the EXAMPLE.

In another preferred embodiment, the compound, or the pharmaceutically acceptable salt, stereoisomer, solvate or prodrug thereof, wherein the compound is selected from the group consisting of

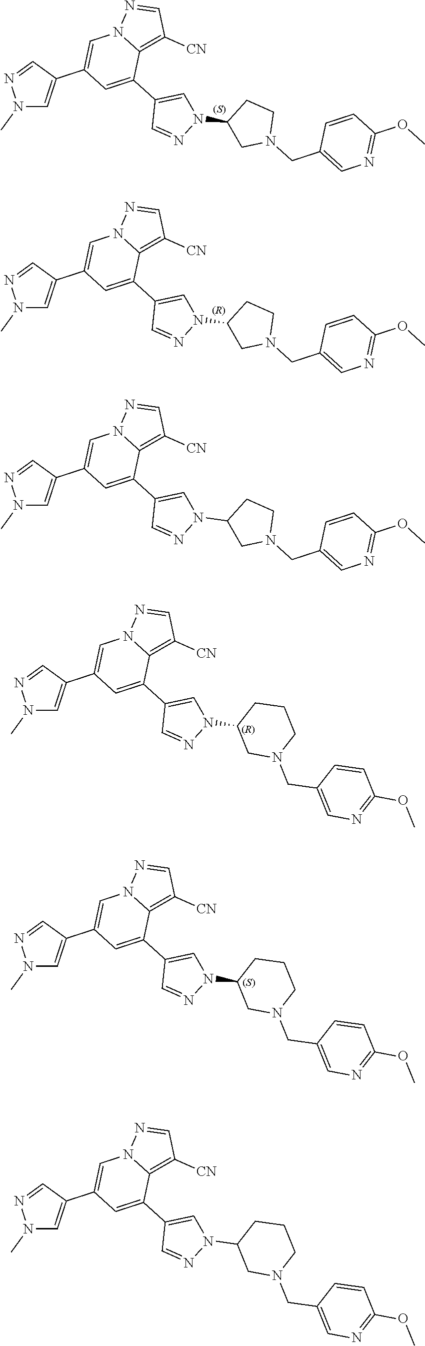

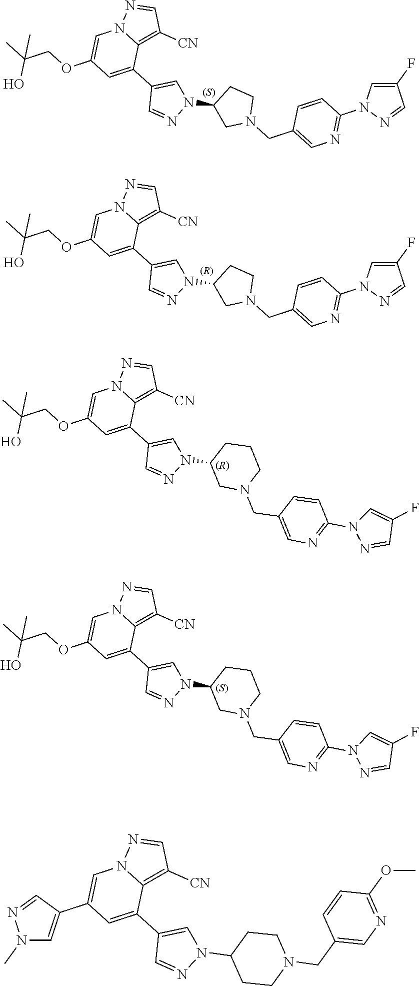

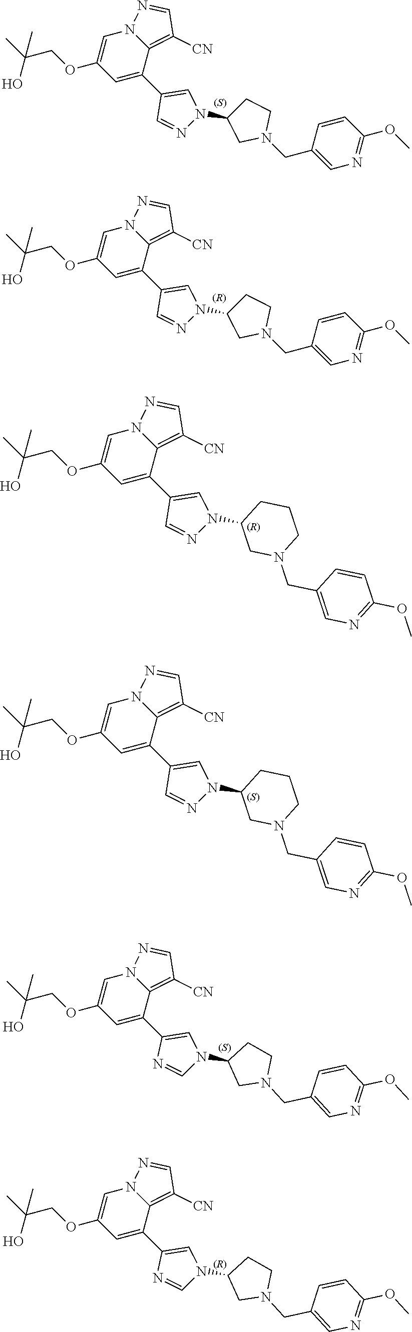

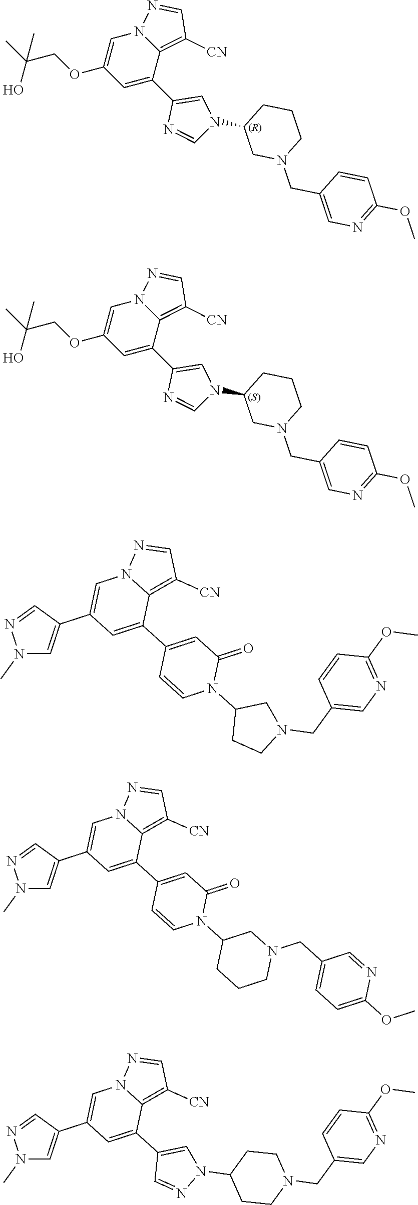

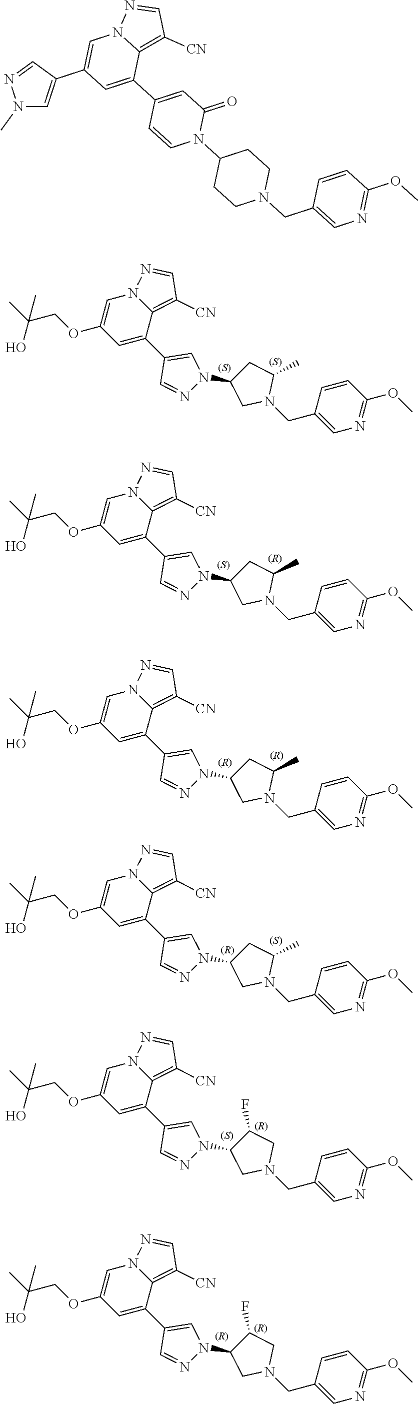

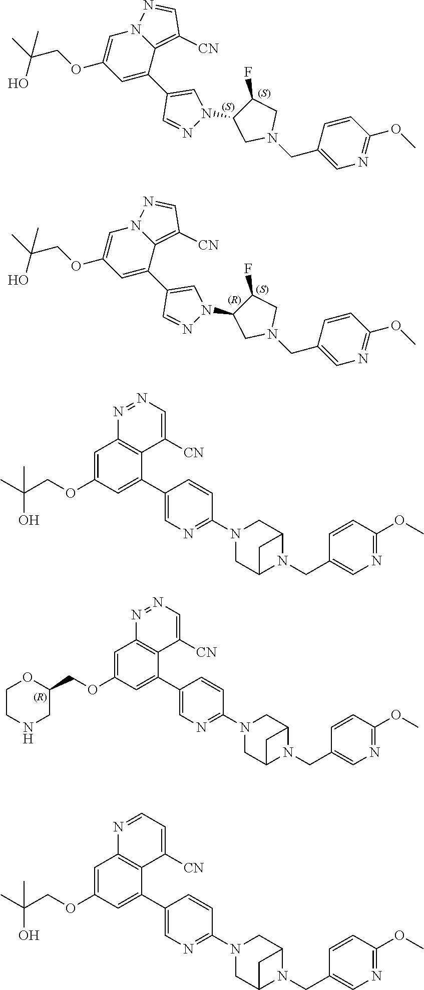

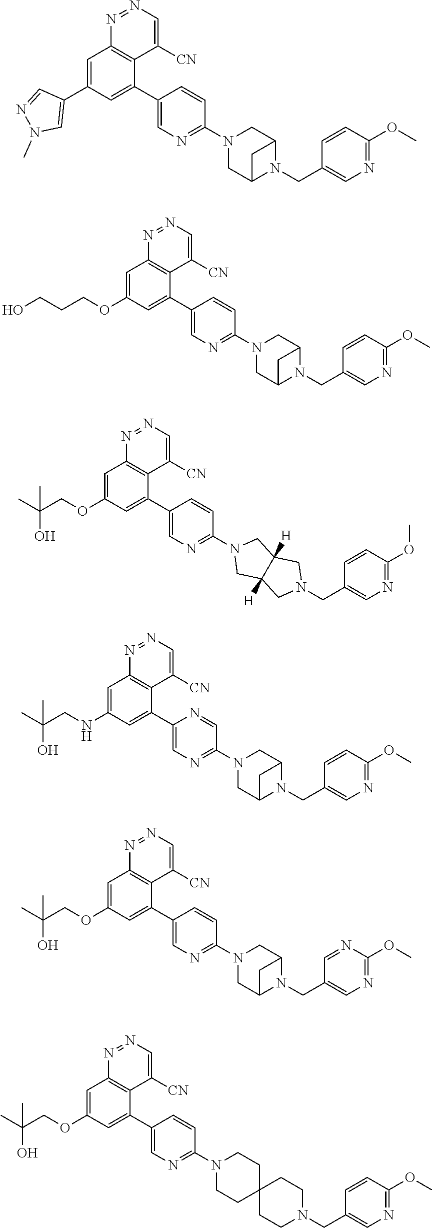

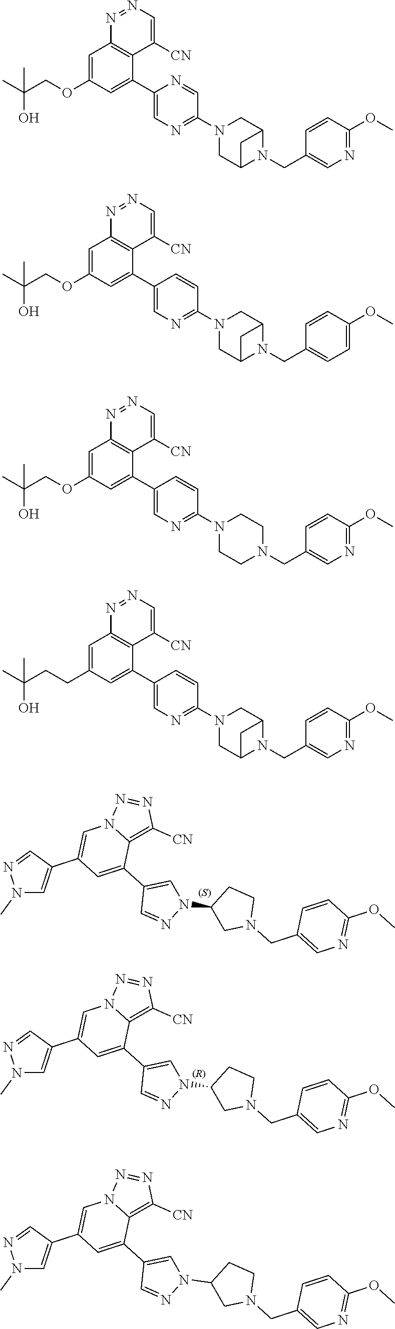

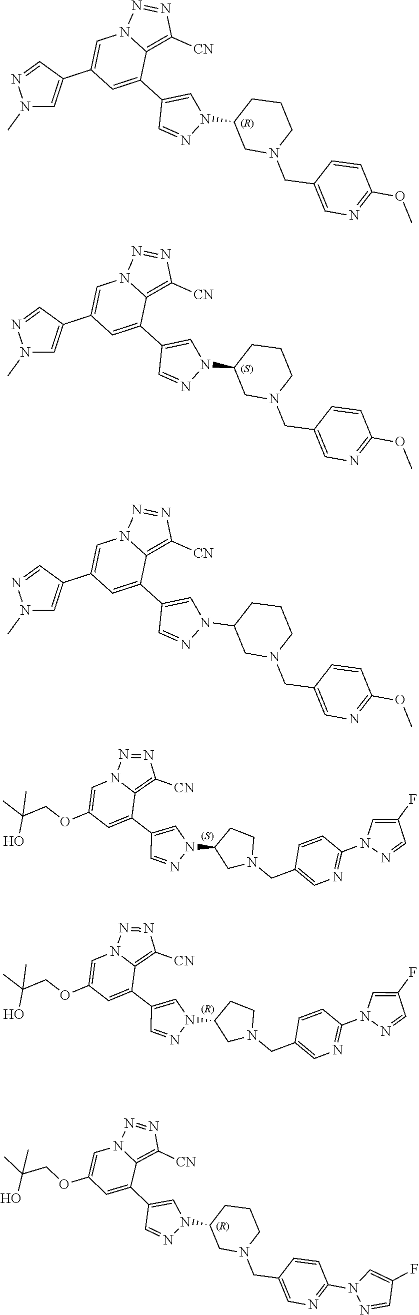

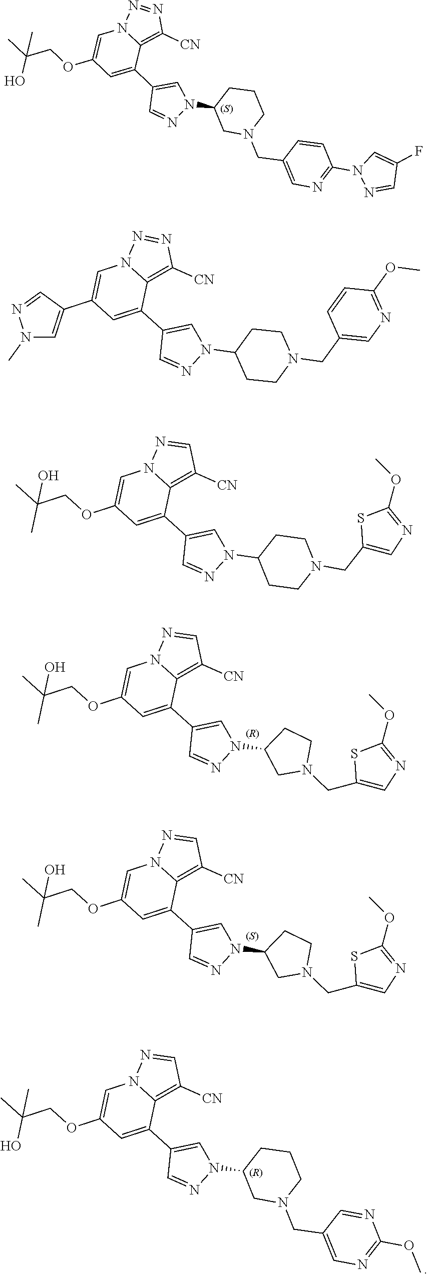

In another preferred embodiment, the compound is selected from the compounds shown in the Example.

In the second aspect of the invention, it provides a pharmaceutical composition comprising the compound, or the pharmaceutically acceptable salt, the stereoisomer, the solvate or the prodrug thereof of the first aspect, and a pharmaceutically acceptable carrier.

In another preferred embodiment, the pharmaceutical composition also comprises other cancer therapeutic agents.

In another preferred embodiment, the other cancer therapeutic agents include radiological agents, cytotoxic agents, kinase inhibitors, immune targeting inhibitors, and angiogenesis inhibitors.

In another preferred embodiment, the pharmaceutical composition further comprises:

PD-1 inhibitor (such as nivolumab, pembrolizumab, JS-001, SHR-120, BGB-A317, IBI-308, GLS-010, GB-226, STW204, HX008, HLX10, BAT 1306, AK105, LZM 009 or the biological analogue thereof, etc.), PD-L1 inhibitor (such as durvalumab, atezolizumab, CS1001, KN035, HLX20, SHR-1316, BGB-A333, JS003, CS1003, KL-A167, F 520, GR1405, MSB2311 or the biological analogue thereof, etc.), CD20 antibody (such as rituximab, obinutuzumab, ofatumumab, tositumomab, ibritumomab, etc.), CD47 antibody (such as Hu5F9-G4, CC-90002, TTI-621, TTI-622, OSE-172, SRF-231, AIX-148, NI-1701, SHR-1603, IBI188, IMM01), ALK inhibitor (such as Ceritinib, Alectinib, Brigatinib, Lorlatinib, Ocatinib), PI3K inhibitor (such as Idelalisib, Dactolisib, Taselisib, Buparlisib, etc.). BTK inhibitor (such as Ibrutinib, Tirabrutinib, Acalabrutinib, etc.), EGFR inhibitor (such as Afatinib, Gefitinib, Erlotinib, Lapatinib, Dacomitinib, Icotinib, Canertinib, etc.), VEGFR inhibitor (such as Sorafenib, Pazopanib, Regorafenib, Cabozantinib, Sunitinib, Donafenib, etc.), HDAC inhibitor (such as Givinostat, Droxinostat, Entinostat, Dacinostat, Tacedinaline, etc.), CDK inhibitor (such as Palbociclib, Ribociclib, Abemaciclib, Lerociclib, etc.), MEK inhibitor (such as Selumetinib (AZD6244), Trametinib (GSK1120212), PD0325901, U0126, AS-703026, PD184352 (CI-1040), etc.), Akt inhibitors (such as MK-2206, Ipatasertib, Capivasertib, Afuresertib, Uprosertib, etc.), mTOR inhibitor (such as Vistusertib, etc.). SHP2 inhibitor (such as RMC-4630, JAB-3068, TNO 155, etc.), IGF-1R inhibitor (such as Ceritinib, Okatinib, Linsitinib, BMS-754807, GSK1838705A, etc.) or combinations thereof.

In the third aspect of the invention, it provides a use of the compound, the pharmaceutically acceptable salt, stereoisomer, solvate or prodrug thereof of the first aspect or the pharmaceutical composition of the second aspect in the preparation of a medicament for inhibiting RET kinase activity in a cell or a subject.

In another preferred embodiment, the compound of the first aspect or the pharmaceutical composition of the second aspect is used for the preparation of a medicament for the treatment of RET-related cancer.

In another preferred embodiment, the RET-related cancer is a cancer characterized by a disorder in the expression or activity level of the RET gene, the RET kinase protein or any of the same proteins.

In another preferred embodiment, RET-related cancer is selected from the group consisting of lung cancer, papillary thyroid carcinoma, medullary thyroid carcinoma, differentiated thyroid carcinoma, recurrent thyroid carcinoma, refractory differentiated thyroid carcinoma, multiple 2A or 2B endocrine tumors (MEN2A or MEN2B respectively), pheochromocytoma, parathyroid hyperplasia, breast cancer, colorectal cancer, papillary renal cell carcinoma, gastrointestinal mucosal ganglioneuroma and cervical cancer.

In another preferred embodiment, the medicament is a medicament for treating a subject with developed resistance to cancer treatment.

In another preferred embodiment, the medicament is a medicament for treating a subject suffering from a condition mediated by abnormal RET activity.

In another preferred embodiment, the cells are mammalian cells.

In another preferred embodiment, the subject is a mammal, preferably a human.

In the fourth aspect of the invention, it provides a method for inhibiting RET kinase activity in a cell or a subject, the method comprises the step of contacting the cell or administering to the subject the compound of the first aspect or the pharmaceutical composition of the second aspect.

In the fifth aspect of the invention, it provides a method for treating a subject suffering from a condition mediated by abnormal RET activity, the method comprises administering to the subject a therapeutically effective amount of the compound of the first aspect or the pharmaceutical composition of the second aspect.

In the sixth aspect of the present invention, it provides a method for treating a subject resistant to cancer treatment, the method comprises administering to the subject a therapeutically effective amount of the compound of the first aspect or the pharmaceutical composition of the second aspect.

It should be understood that in the present invention, any of the technical features specifically described above and below (such as in the Example) can be combined with each other, thereby constituting new or preferred technical solutions. Limited by space, it will not be repeated here.

### DETAILED DESCRIPTION OF THE INVENTION

### Terms

In the present invention, unless otherwise specified, the terms used have the general meanings known to those skilled in the art.

The term “C1-C6 alkyl” refers to a linear or branched alkyl, including 1-6 carbon atoms such as methyl, ethyl, propyl, isopropyl

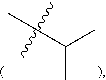 [SMILES: *C(C)C]

n-butyl, tert-butyl, isobutyl (such as

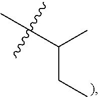 [SMILES: *C(C)CC]

n-pentyl, isopentyl, n-hexyl, isohexyl. “Substituted alkyl” refers to one or more positions in the alkyl are substituted, especially 1-4 substituents, which can be substituted at any position. As used herein, “alkyl” includes “substituted alkyl”. Typical substituents include, but are not limited to one or more of the following groups: such as hydrogen, deuterium, halogen (such as monohalogenated substituent or polyhalogenated substituents, and the latter such as trifluoromethyl or alkyl containing Cl₃), cyano, nitro, oxo (═O), trifluoromethyl, trifluoromethoxy, cycloalkyl, alkenyl, cycloalkenyl, alkynyl, heterocycle, aromatic ring, ORₐ, SRₐ, S(═O)R_{c}, S(═O)₂R_{c}, P(═O)₂R_{c}, S(═O)₂OR_{c}, P(═O)₂OR_{c}, NR_{b}R_{c}, NR_{b}S(═O)₂R_{c}, NR_{b}P(═O)₂R_{c}, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)OR_{c}, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ or NR_{b}P(═O)₂R_{c}, wherein Rₐ can independently represent hydrogen, deuterium, alkyl, cycloalkyl, alkenyl, cycloalkenyl, alkynyl, heterocycle or aromatic ring, R_{b}, R_{c} and R_{d} can independently represent hydrogen, deuterium, alkyl, cycloalkyl, heterocycle or aromatic ring, or R_{b} and R_{c} together with the N atom form a heterocycle, R_{c} can independently represent hydrogen, alkyl, cycloalkyl, alkenyl, cycloalkenyl, alkynyl, heterocycle or aromatic ring. The above typical substituents, such as alkyl, cycloalkyl, alkenyl, cycloalkenyl, alkynyl, heterocyclic or aromatic ring can be optionally substituted.

The term “heteroalkyl” refers to a group in an alkyl whose carbon atoms are substituted by atoms selected from, e.g., oxygen, nitrogen, sulfur, phosphorus, or combinations thereof. Numeric ranges may be given for example C1-C6 heteroalkyl refers to the number of carbons in the chain, and it includes 1 to 6 carbon atoms in the example. For example, —CH₂OCH₂CH₃ is called as “C₃” heteroalkyl. The connection with the rest of the molecule can be through heteroatoms or carbons in heteroalkyl chain.

The term “3-7 membered ring” means a 3, 4, 5, 6 or 7 membered ring and includes a saturated ring and an unsaturated ring, and the saturated ring includes cycloalkyl or heterocycloalkyl (containing 1-3 heterocyclic atoms of N, O and S), and the cycloalkyl includes cyclopropyl, cyclobutyl, cyclopentyl, cyclohexyl, cycloheptyl; heterocycloalkyl includes azacyclopropyl, azacyclobutyl, azacyclopentyl azacyclohexyl, oxacyclopropyl, oxacyclobutyl, oxacyclopentyl, oxacyclohexyl, etc. Unsaturated ring includes cyclohexenyl, cyclohexadienyl, cyclopentenyl, etc.

The term “5 or 6-membered aromatic group” includes 5-membered heteroaryl, 6-membered heteroaryl and phenyl.

In the present invention, the 5- or 6-membered heteroaryl in Ar₁ can be

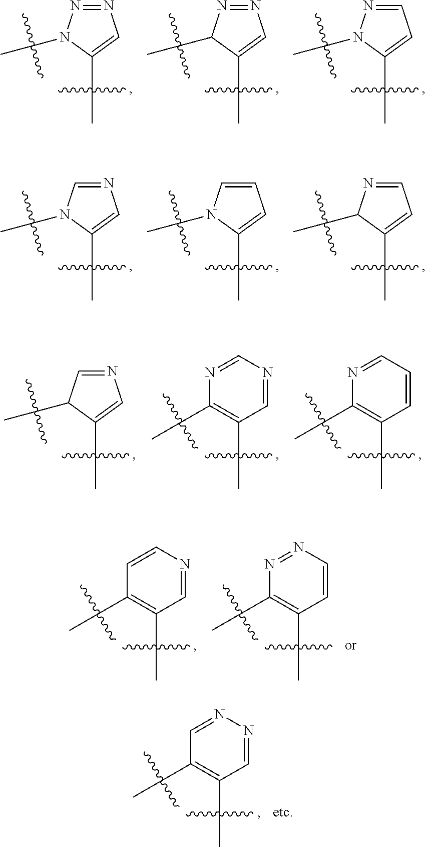

In the resent invention, the 5- or 6-membered heteroaryl in Ar₂ can be

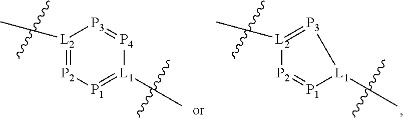

wherein, P₁, P₂, P₃ and P₄ are each independently selected from N or CH, wherein 0, 1 or 2 of P₁, P₂, P₃ and P₄ are N, L₁ and L₂ are each independently selected from N or C.

The term “alkylene” refers to a group formed by “alkyl” removing a hydrogen atom such as methylene, ethylidene, propylidene, and isopropylidene (such as

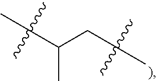 [SMILES: *CC(*)C]

butylidene such as

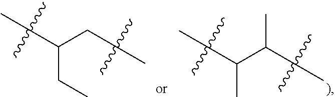

pentylidene (such as

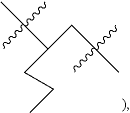 [SMILES: *CC(*)CCC]

hexylidene (such as or

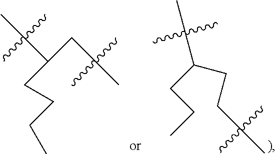

heptylidene (such as

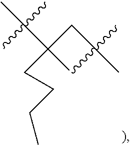 [SMILES: *CC(*)(C)CCCC]

etc.

The term “cycloalkyl” refers to a fully saturated cyclic hydrocarbon group, and “C3-C6 cycloalkyl” and “C3-C12 cycloalkyl” refer to containing 3-6 and 3-12 carbon atoms, respectively. “Substituted cycloalkyl” refers to one or more positions in the cycloalkyl are substituted, especially 1-4 substituents, which can be substituted at any position. Typical substituents include, but are not limited to one or more of the following groups: such as hydrogen, deuterium, halogen (such as monohalogenated substituent or polyhalogenated substituents, and the latter such as trifluoromethyl or alkyl containing Cl₃), cyano, nitro, oxo (═O), trifluoromethyl, trifluoromethoxy, cycloalkyl, alkenyl, cycloalkenyl, alkynyl, heterocycle, aromatic ring, ORₐ, SRₐ, S(═O)R_{c}, S(═O)₂R_{c}, P(═O)₂R_{c}, S(═O)₂OR_{c}, P(═O)₂OR_{c}, NR_{b}R_{c}, NR_{b}S(═O)₂R_{c}, NR_{b}P(═O)₂R_{c}, S(═O)₂NR_{b}R_{c}, P(═O)₂NR_{b}R_{c}, C(═O)OR_{d}, C(═O)Rₐ, C(═O)NR_{b}R_{c}, OC(═O)R^{a}, OC(═O)NR_{b}R_{c}, NR_{b}C(═O)OR_{c}, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ or NR_{b}P(═O)₂R_{c}, wherein Rₐ can independently represent hydrogen, deuterium, alkyl, cycloalkyl, alkenyl, cycloalkenyl, alkynyl, heterocycle or aromatic ring, R_{b}, R_{c} and R_{d} can independently represent hydrogen, deuterium, alkyl, cycloalkyl, heterocycle or aromatic ring, or R_{b} and R_{c} together with the N atom form a heterocycle. R_{c} can independently represent hydrogen, deuterium, alkyl, cycloalkyl, alkenyl, cycloalkenyl, alkynyl, heterocycle or aromatic ring. The above typical substituents can be optionally substituted. Typically substituent also includes spiro, bridged or fused ring, especially spiro cycloalkyl, spiro cycloalkenyl, spiro heterocyclyl (excluding heteroaryl ring), bridged cycloalkyl, bridged cycloalkenyl, bridged heterocyclyl (excluding heteroaryl ring), fused cycloalkyl, fused cycloalkenyl, fused heterocyclyl or fused aryl, the above cycloalkyl, cycloalkenyl, heterocyclyl and heteroaryl can be optionally substituted.

The term “heterocyclyl” refers to a completely saturated or partially unsaturated cyclic group (including but not limited to, for example, 4-7-membered monocyclic, 7-11-membered spiro heterocyclyl, 7-8 membered bridged heterocyclyl), wherein at least one heteroatom is present in a ring having at least one carbon atom. Each heterocyclyl containing heteroatom can have 1, 2, 3 or 4 heteroatoms, and these heteroatoms are selected from nitrogen atom, oxygen atom or sulfur atom, wherein the nitrogen atom or sulfur atom can be oxidized, and the nitrogen atom can also be quaternized. Heterocyclyl can be attached to the residue of any heteroatom or carbon atom of the ring or ring molecule. Typical monocyclic heterocyclyls include, but are not limited to azacyclobutyl, pyrrolidyl, oxacyclobutyl, pyrazolinyl, imidazolinyl, imidazolidinyl, oxazolidinyl, isoxazolidinyl, thiazolidinyl, isothiazolidinyl, tetrahydrofuryl, piperidyl, piperazinyl, 2-oxopiperazinyl, 2-oxo piperidyl, 2-oxopyrrolidyl, hexahydroazepinyl, 4-piperidinone, tetrahydropyranyl, morpholinyl, thiomorpholinyl, thiomorpholinylsulfoxide, thiomorpholinylsulfone, 1,3-dioxane and tetrahydro-1,1-dioxythienyl, etc. The polycyclic heterocyclyl includes spiro, fused, and bridged heterocyclyls; the spiro, fused, and bridged heterocyclyls involved are optionally connected with other groups by single bond, or are further fused with other cycloalkyl, heterocyclyl, aryl and heteroaryl by any two or more atoms of the ring. The heterocyclyl may be substituted or unsubstituted, when being substituted, the substituent is preferably one or more of the following groups, independently selected from alkyl, deuterated alkyl, haloalkyl, alkoxy, haloalkoxy, alkenyl, alkynyl, alkylthio, alkylamino, halogen, amino, nitro, hydroxy, thiol, cyano, cycloalkyl, heterocyclyl, aryl, heteroaryl, cycloalkylthio, oxo, carboxy, and ester.

The term “aryl” refers to an aromatic cyclic hydrocarbon group, and “C6-C14 aryl” refers to an aryl containing 6-14 carbon atoms, especially monocyclic and bicyclic groups such as phenyl, biphenyl or naphthyl. Any aromatic ring having two or more aromatic rings (bicyclic, etc.), the aromatic rings of aryl may be connected by single bond (such as biphenyl) or fused (such as naphthalene, anthracene, etc.). “Substituted aryl” refers to one or more positions in the aryl are substituted, especially 1-3 substituents, which can be substituted at any position. Typical substituents include, but are not limited to one or more of the following groups: such as hydrogen, deuterium, halogen (such as monohalogenated substituent or polyhalogenated substituents, and the latter such as trifluoromethyl or alkyl containing Cl₃), cyano, nitro, oxo (═O), trifluoromethyl, trifluoromethoxy, cycloalkyl, alkenyl, cycloalkenyl, alkynyl, heterocycle, aromatic ring, ORₐ, SRₐ, S(═O)R_{c}, S(═O)₂R_{c}, P(═O)₂R_{c}, S(═O)₂OR_{c}, P(═O)₂OR_{c}, NR_{b}R_{c}, NR_{b}S(═O)₂R_{c}, NR_{b}P(═O)₂R_{c}, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NRC(═O)OR_{c}, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ or NR_{b}P(═O)₂R_{c}, wherein Rₐ can independently represent hydrogen, deuterium, alkyl, cycloalkyl, alkenyl, cycloalkenyl, alkynyl, heterocycle or aromatic ring, R_{b}, R_{c} and R_{d} can independently represent hydrogen, deuterium, alkyl, cycloalkyl, heterocycle or aromatic ring, or R_{b} and R_{c} together with the N atom form a heterocycle, R_{c} can independently represent hydrogen, deuterium, alkyl, cycloalkyl, alkenyl, cycloalkenyl, alkynyl, heterocycle or aromatic ring. The above typical substituents can be optionally substituted. Typical substituent also includes fused ring, especially fused cycloalkyl, fused cycloalkenyl, fused heterocyclyl or fused aryl, the above cycloalkyl, cycloalkenyl, heterocyclyl and heteroaryl can be optionally substituted.

The term “5-14 membered heteroaryl” refers to a heteroaromatic system containing 1-4 heteroatoms, 5-14 ring atoms, wherein the heteroatom is selected from oxygen, nitrogen and sulfur. The heteroaryl is preferably 5 to 10 membered ring, more preferably 5 or 6 membered, such as pyrryl, pyrazolyl, imidazolyl, oxazolyl, isoxazolyl, thiazoyl, thiadiazolyl, isothiazolyl, furanyl, pyridyl, pyrazinyl, pyrimidyl, pyridazinyl, triazinyl, triazolyl, and tetrazolyl, etc. The “heteroaryl” may be substituted or unsubstituted, when being substituted, the substituent is preferably one or more of the following groups, independently selected from alkyl, deuterated alkyl, haloalkyl, alkoxy, haloalkoxy, alkenyl, alkynyl, alkylthio, alkylamino, halogen, amino, nitro, hydroxy, thiol, cyano, cycloalkyl, heterocyclyl, aryl, heteroaryl, cycloalkylthio, oxo, carboxy, and ester.

The term “C1-C6 alkoxyl” refers to a straight, branched chain or cyclic alkoxyl having from 1 to 6 carbon atoms, including but not limited to methoxyl, ethoxyl, propoxyl, isopropoxyl and butoxyl, etc. Preferably C1-C3 alkoxy. “Alkoxyalkyl” refers to the hydrogen atom in an alkyl is substituted by alkoxy, such as CH₃OCH₂— and CH₃OCH₂CH₂—.

The term “halogen” or “halo” is chlorine, bromine, fluorine, and iodine.

The term “deuterated” refers to substituted by deuterium.

The term “hydroxy” refers to a group with a structure of OH.

The term “nitro” refers to a group with a structure of NO₂.

The term “cyano” refers to a group with a structure of CN.

The term “ester” refers to a group with a structure of —COOR, wherein R represents hydrogen, C1-C6 alkyl or substituted C1-C6 alkyl, C3-C8 cycloalkyl or substituted C3-C8 cycloalkyl, C4-C10 cycloalkenyl or substituted C4-C10 cycloalkenyl, aryl or substituted C6-C14 aryl, 3-8 membered heterocyclyl or substituted heterocyclyl.

The term “amino” refers to a group with a structure of —NRR′, wherein R and R′ can independently represent hydrogen. C1-C6 alkyl or substituted C1-C6 alkyl, C3-C8 cycloalkyl (preferably C3-C6 cycloalkyl) or substituted C3-C8 cycloalkyl (preferably C3-C6 cycloalkyl), C4-C10 cycloalkenyl or substituted C4-C10 cycloalkenyl, aryl or substituted C6-C14 aryl, 3-8 membered heterocyclyl or substituted heterocyclyl. R and R′ may be the same or different in the dialkylamino segment. “C1-C6 alkylamino” and “C3-C8 cycloalkylamino” are C1-C6 alkyl NH—. C3-C8 cycloalkyl NH—, respectively.

The term “amido” refers to a group with a structure of —CONRR′, wherein R and R′ independently represent hydrogen, C1-C6 alkyl or substituted C1-C6 alkyl, C3-C8 cycloalkyl or substituted C3-C8 cycloalkyl, C4-C10 cycloalkenyl or substituted C4-C10 cycloalkenyl, aryl or substituted C6-C14 aryl, 3-8 membered heterocyclyl or substituted heterocyclyl. R and R′ may be the same or different in the dialkylamino segment.

The term “sulfonamido” refers to a group with a structure of —SO₂NRR′, wherein R and R′ independently represent hydrogen, C1-C6 alkyl or substituted C1-C6 alkyl. C3-C8 cycloalkyl or substituted C3-C8 cycloalkyl, C4-C10 cycloalkenyl or substituted C4-C10 cycloalkenyl, aryl or substituted C6-C14 aryl, 3-8 membered heterocyclyl or substituted heterocyclyl. R and R′ may be the same or different in the dialkylamino segment.

The term “C6-C14 aryl C1-C6 alkyl” refers to the hydrogen atom in C1-C6 alkyl is substituted by C6-C14 aryl, such as benzyl, phenylethyl, etc.

In the present invention, the term “substituted” refers to the substitution of one or more hydrogen atoms on a specific group by specific substituent. The specific substituents are those described in the preceding paragraph or those present in each Example. Unless otherwise specified, a substituted group may have a substituent selected from a specific group at any substitutable position of the group, and the substituent may be the same or different in each position. In the present invention, the groups (e.g. alkyl, alkylene, cycloalkyl, heteroalkyl, heterocyclyl, aryl, heteroaryl, etc.) include the H-substituted groups on their corresponding groups. Those skilled in the art should understand that the combinations of substituents contemplated by the present invention are those that are stable or chemically achievable. The substituent is such as (but is not limited to): halogen, hydroxy, cyano, carboxy (—COOH), C1-C6 alkyl, C2-C6 alkenyl, C2-C6 alkynyl, C3-C8 cycloalkyl, 3- to 12 membered heterocyclyl, aryl, heteroaryl, C1-C8 aldehyde, C2-C10 acyl, C2-C10 ester, amino, C1-C6 alkoxy, C1-C10 sulfonyl, and C1-C6 uramido, etc.

In the present invention, more (multiple) generally refers to more than two.

Unless otherwise stated, it is assumed that any heteroatom with a lower valence state has enough hydrogen atoms to replenish its valence state.

When the substituent is a non-terminal substituent, it is a subunit of the corresponding substituent, such as alkyl corresponding to alkylene, cycloalkyl corresponding to cycloalkylene, heterocyclyl corresponding to heterocyclylene, alkoxy corresponding to alkyleneoxy, etc.

Active Ingredient

As used herein, “the compound of the present invention” refers to the compound represented by the formula F, and further comprises the stereoisomer or optical isomer, pharmaceutically acceptable salt, prodrug or solvate of the compound of formula F.

The compound of formula F has the following structure:

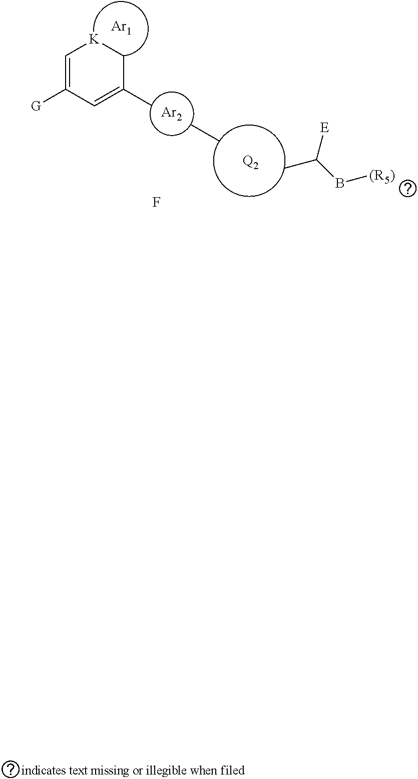

wherein, G, K, Ar₁, Ar₂, Q₂, B, E, R₅ and f are defined as above.

Preferably, in formula F, Ar₁ is substituted or unsubstituted group consisting of

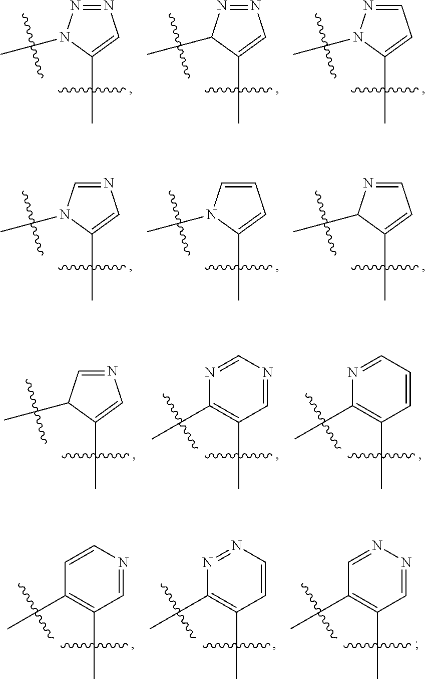

wherein the “substituted” means being substituted by one or more groups selected from the group consisting of H, CN, halogen, methyl, ethyl and cyclopropyl;

Ar₂ is a substituted or unsubstituted 5-membered heteroaryl, wherein the “substituted” means being substituted by one or more groups selected from the group consisting of C1-C6 alkyl, halogen, hydroxyl, oxo (═O), C1-C6 heteroalkyl, C1-C6 alkoxy, C3-C14 cycloalkyl, 3-14-membered heterocycloalkyl and cyano;

Q₂ is a saturated 5-6-membered monocyclic heterocyclyl containing one or two nitrogen ring heteroatoms, and the H on Q₂ may optionally be substituted by one or more substituents selected from the group consisting of deuterium, hydroxy, halogen, cyano, ester, amido, oxo, carbonyl, amino, C1-C6 alkyl, C1-C6 haloalkyl, C1-C6 thioalkyl, C1-C6 alkoxy, C1-C6 heteroalkyl, C1-C6 alkylamino, C3-C6 cycloalkyl, C3-C8 cycloalkylamino, C6-C14 aryl and 5-14-membered heteroaryl;

B is a 5-6-membered heteroaryl and the H on B may optionally be substituted by one or more substituents selected from the group consisting of deuterium, hydroxy, halogen, cyano, ester, amido, carbonyl, amino, C1-C6 alkyl, C1-C6 haloalkyl, C1-C6 thioalkyl, C1-C6 alkoxy, C1-C6 heteroalkyl, C1-C6 alkylamino, C3-C6 cycloalkyl, C3-C8 cycloalkylamino, C6-C14 aryl and 5-14-membered heteroaryl;

E is hydrogen or substituted or unsubstituted C1-C6 alkyl, wherein the “substituted” means being substituted by 0-5 Rₐ, R^{a} is defined as above.

Preferably, Q2 is

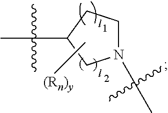

wherein, l₁ and l₂ are each independently 0, 1, 2 or 3, and l₁+l₂ is an integer of 1-4:

y is 0, 1, 2 or 3:

Rn is selected from deuterium, hydroxy, halogen, cyano, ester, amido, carbonyl, oxo (═O), amino, C1-C6 alkyl, C1-C6 haloalkyl, C1-C6 thioalkyl, C1-C6 alkoxy, C1-C6 heteroalkyl, C1-C6 alkylamino, C3-C6 cycloalkyl, C3-C8 cycloalkylamino, C6-C14 aryl or 5-14-membered heteroaryl.

Preferably, in formula F,

Ar₁ is substituted or unsubstituted group consisting of

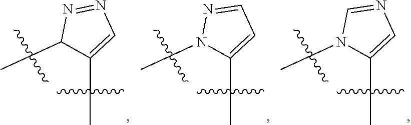 [SMILES: *C1=CN=NC1* ; *c1ccnn1* ; *c1cncn1*]

wherein the “substituted” means being substituted by one or more groups selected from the group consisting of H, CN, halogen, methyl, ethyl and cyclopropyl;

Ar₂ is selected from

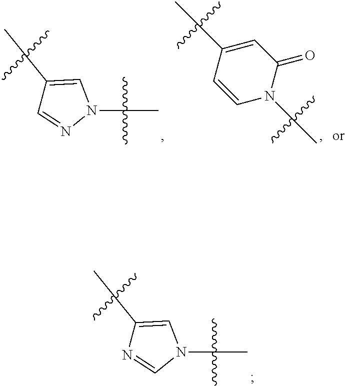 [SMILES: *c1cnn(*)c1 ; *c1ccn(*)c(=O)c1 ; *c1cn(*)cn1]

G is selected from

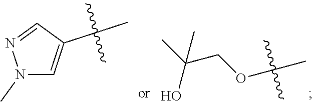

R₅ is selected from C1-C3 alkoxy or

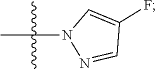 [SMILES: *n1cc(F)cn1]

preferably, R₅ is selected from methoxy or

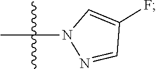 [SMILES: *n1cc(F)cn1]

Q₂ is selected from

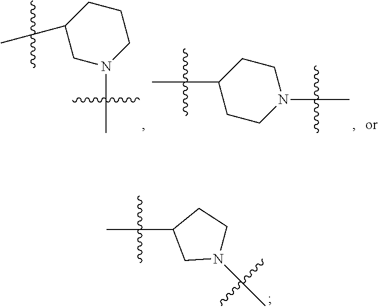 [SMILES: *C1CCCN(*)C1 ; *C1CCN(*)CC1 ; *C1CCN(*)C1]

B is substituted or unsubstituted group consisting of pyridyl, pyrimidinyl and thiazolyl; wherein the “substituted” means being substituted by one or more substituents selected from the group consisting of deuterium, hydroxy, halogen, cyano, ester, amido, carbonyl, amino, C1-C6 alkyl, C1-C6 haloalkyl, C1-C6 thioalkyl, C1-C6 alkoxy, C1-C6 heteroalkyl, C1-C6 alkylamino, C3-C6 cycloalkyl, C3-C8 cycloalkylamino, C6-C14 aryl and 5-14-membered heteroaryl;

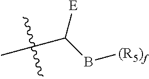

moiety is selected from

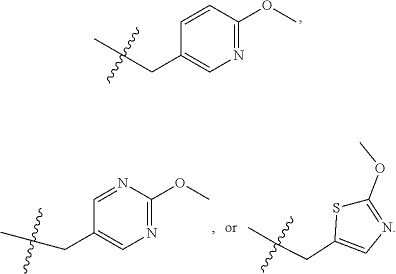

The salt of the compound in the present invention that may be formed are also within the scope of the present invention. Unless otherwise stated, the compound in the present invention is understood to include its salt. The term “salt” as used herein refers to a salt formed in the form of acid or base from inorganic or organic acid and base. Further, when the compound in the present invention contains a base fragment, it includes, but is not limited to pyridine or imidazole, when it contains an acid segment, it includes, but is not limited to carboxylic acid. The zwitter-ion that may be formed (“inner salt”) is included within the scope of the term “salt”. Pharmaceutically acceptable (i.e., non-toxic, physiologically acceptable) salt is preferred, although other salts are also useful and may be used, for example, in the separation or purification steps of the preparation process. The compound of the present invention may form a salt, for example, formula F is reacted with a certain amount (such as an equivalent amount) of an acid or base, and precipitated in a medium, or freeze-dried in aqueous solution.

The base fragment contained in the compounds of the present invention includes but is not limited to amines or pyridine or imidazole rings, may form salt with organic or inorganic acid. Typical acids that form salts include acetate (such as acetate or trihalogenated acetic acid, such as trifluoroacetic acid), adipate, alginate, ascorbate, aspartate, benzoate, benzene sulfonate, disulfate, borate, butyrate, citrate, camphorate, camphor sulfonate, cyclopentane propionate, diethylene glycolate, lauryl sulfate, ethanesulphonate, fumarate, gluceptate, glycerophosphate, hemisulphate, enanthate, caproate, hydrochloride, hydrobromide, hydriodate, isethionate (e.g., 2-hydroxy-ethesulfonate), lactate, maleate, mesylate, naphthalenesulfonate (e.g., 2-naphthalenesulfonate), nicotinate, nitrate, oxalate, pectate, persulfate, phenylpropionate (e.g., 3-phenylpropionate), phosphate, picrate, pivalate, propionate, salicylate, succinate, sulfate (e.g., formed with sulfuric acid), sulfonate, tartrate, thiocyanate, toluenesulfonate (e.g., tosilate), dodecanoate, etc.

Some compounds of the invention may contain acidic fragments including, but not limited to carboxylic acid which may form salts with various organic or inorganic bases. Typical salt formed by base includes ammonium salt, alkali metal salt (such as sodium, lithium and potassium salts), alkaline earth metal salt (such as calcium and magnesium salts), and salt formed by organic bases (such as organic amines), such as benzathine, dicyclohexylamine, hydrabamine (salt formed with N,N-bis (dehydroabietyl) ethylenediamine), N-methyl-D-glucanamine, N-methyl-D-glucoamide, tert-butylamine, and the salt formed with amino acids such as arginine, lysine, etc. Basic nitrogen-containing groups can form quaternary ammonium salts with halides, such as small molecular alkyl halides (such as chlorides, bromides and iodides of methyl, ethyl, propyl and butyl), dialkyl sulfate (such as dimethyl, diethyl, dibutyl, and dipentyl sulfates), long chain halides (such as chlorides, bromides and iodides of decyl, dodecyl, tetradecyl, and tetradecyl), aralkyl halides (such as bromides of benzyl and phenyl), etc.

The prodrug and solvate of the compound in the present invention are also included within the scope of the present invention. The term “prodrug” herein refers to a compound which will produce a compound, salt, or solvate of the present invention after chemical transformation of a metabolic or chemical process when it is used in the treatment of an associated disease. The compounds of the invention include solvates such as hydrates.

Compound, salt or solvate in the present invention, may be present in tautomeric forms such as amide and imino ether. All of these tautomers are part of the present invention.

Stereoisomers of all compounds (e.g., those asymmetric carbon atoms that may be present due to various substitutions), including their enantiomeric forms and non-enantiomed forms, all belong to the protection scope of the present invention. The independent stereoisomer in the present invention may not coexist with other isomers (e.g., as a pure or substantially pure optical isomer with special activity), or may be a mixture (e.g., racemate), or a mixture formed with all other stereoisomers or a part thereof. The chiral center of the present invention has two configurations of S or R, which is defined by International Union of Pure and Applied Chemistry (IUPAC) in 1974. The racemization form can be solved by physical methods, such as fractional crystallization, or separation crystallization by derivation into diastereomers, or separation by chiral column chromatography. Individual optical isomer can be obtained from racemate by appropriate methods, including but not limited to conventional methods, such as recrystallization after salting with optically active acids.

Weight content of compound in the present invention obtained by preparation, separation and purification in turn is equal to or greater than 90%, such as equal to or greater than 95%, equal to or greater than 99% (“very pure” compound), which is listed in the description of the text. In addition, the “very pure” compound of the present invention is also part of the present invention.

All configuration isomers of the compound of the present invention are within the scope, whether in mixture, pure or very pure form. The definition of the compound of the present invention comprises cis (Z) and trans (E) olefin isomers, and cis and trans isomers of carbocycle and heterocycle.

In the entire specification, the groups and substituents can be selected to provide stable fragments and compounds.

Specific functional groups and chemical term definitions are described in detail below. For the purposes of the present invention, the chemical elements are consistent with Periodic Table of the Elements, CAS version, Handbook of Chemistry and Physics, 75^{th} Ed. The definition of a particular functional group is also described therein. In addition, the basic principles of Organic Chemistry as well as specific functional groups and reactivity are described in “Organic Chemistry”. Thomas Sorrell, University Science Books, Sausalito: 1999, the entire content of which is incorporated herein by reference.

Some compounds of the present invention may exist in specific geometric or stereoisomer forms. The present invention covers all compounds, including their cis and trans isomers, R and S enantiomers, diastereomers, (D) type isomers, (L) type isomers, racemic mixtures and other mixtures. In addition, asymmetric carbon atom can represent substituent, such as alkyl. All isomers and mixtures thereof are included in the present invention.

According to the invention, mixtures of isomers may contain a variety ratios of isomers. For example, mixtures with only two isomers may have the following combinations: 50:50, 60:40, 70:30, 80:20, 90:10, 95:5, 96:4, 97:3, 98:2, 99:1, or 100:0, all ratios of the isomers are within the scope of the present invention. Similar ratios readily understood by those of ordinary skill in the art and ratios for mixtures of more complex isomers are also within the scope of the present invention.

The invention also includes isotope labeled compounds, which are equivalent to the original compounds disclosed herein. However, in practice, it usually occurs when one or more atoms are replaced by atoms with a different atomic weight or mass number. Examples of compound isotopes that may be listed in the present invention include hydrogen, carbon, nitrogen, oxygen, phosphorus, sulfur, fluorine and chlorine isotopes, such as ²H, ³H, ¹³C, ¹¹C, ¹⁴C, ¹⁵N, ¹⁸O, ¹⁷O, ³¹P, ³²P, ³⁵S, ¹⁸F and ³⁶Cl. Compound, or enantiomer, diastereomer, isomer, or pharmaceutically acceptable salt or solvate, the above compound containing isotopes or other isotope atoms are all within the scope of the invention. Some isotope-labeled compounds in the present invention, such as the radioactive isotopes of ³H and ¹⁴C, are also included and are useful in experiments on the tissue distribution of drugs and substrates. Tritium (³H) and Carbon-14 (¹⁴C), which are relatively easy to prepare and detect are the preferred choice. In addition, heavier isotope substitutions such as deuterium. i.e. ²H, have advantages in certain therapies due to their good metabolic stability, such as increased half-life or reduced dosage in vivo, and thus may be preferred in certain situations. Isotope-labeled compounds can be prepared by conventional methods through replacing non-isotopic reagents with readily available isotope-labeled reagents using the disclosed scheme shown in the Example.

If the synthesis of a specific enantiomer of the compound of the invention is to be designed, it can be prepared by asymmetric synthesis, or derivatized with chiral auxiliary reagent, separating the resulting diastereomeric mixture and removing the chiral auxiliary reagent to obtain a pure enantiomer. In addition, if a molecule contains a basic functional group, such as an amino acid, or an acidic functional group, such as a carboxyl group, a diastereomer salt can be formed with a suitable optically active acids or bases, and it can be separated by conventional means, such as crystallization or chromatography, to obtain a pure enantiomer.

As described herein, the compound in the present invention may be substituted with any number of substituents or functional groups to extend its scope. In general, whether the term “substituted” appears before or after the term “optional”, the general formula that includes substituents in the compound of the present invention means the substitution of a specified structural substituent for a hydrogen radical. When multiple locations in a particular structure are replaced by multiple specific substituents, each location of the substituents can be the same or different. The term “substituted” as used herein includes all substitution that allows organic compounds to be substituted. Broadly speaking, the allowable substituents include non-cyclic, cyclic, branched, non-branched, carbocyclic and heterocyclic, aromatic ring and non-aromatic organic compounds. In the present invention, such as heteroatomic nitrogen, its valence state may be supplemented by a hydrogen substituent or by any permitted organic compound described above. Furthermore, the invention is unintentionally limited to the substituted organic compounds in any way. The present invention considers that a combination of substituents and variable groups is good for the treatment of diseases (such as infectious or proliferative diseases) in the form of stable compounds. The term “stable” herein refers to a stable compound which is sufficient for maintaining the integrity of the compound structure within a sufficiently long time, preferably being effective in a sufficiently long time, which is hereby used for the above purposes.

The metabolites of the compounds of the present application and their pharmaceutically acceptable salts, and prodrugs that can be converted into the compounds of the present application and their pharmaceutically acceptable salts thereof in vivo, are also included in the claims.

Active Ingredient

As used herein, the term “compound of the invention” refers to the compound of formula F. The term also includes its pharmaceutically acceptable salt, stereoisomer, solvate or prodrug.

The term “pharmaceutically acceptable salt” refers to a salt formed by a compound of the present invention and an acid or a base suitable for use as a medicine. Pharmaceutically acceptable salt comprises inorganic salt and organic salt. A preferred class of salt is a salt formed by a compound of the present invention with an acid. Acid suitable for salt formation including but not limited to inorganic acids such as hydrochloric acid, hydrobromic acid, hydrofluoric acid, sulfuric acid, nitric acid, phosphoric acid, etc.; organic acids such as formic acid, acetic acid, trifluoroacetic acid, propionic acid, oxalic acid, malonic acid, succinic acid, fumaric acid, maleic acid, lactic acid, malic acid, tartaric acid, citric acid, picric acid, benzoic acid, mesylate, ethanesulfonic acid, p-toluenesulfonic acid, benzenesulfonic acid, naphthalenesulfonic acid, etc.; and amino acid such as proline, phenylalanine, aspartic acid and glutamic acid, etc. Another class of preferred salt is the salt formed by the compound of the invention with base, such as alkali metal salt (e.g. sodium or potassium salt), alkaline earth metal salt (e.g. magnesium or calcium salt), ammonium salt (e.g. lower alkanol ammonium salt and other pharmaceutically acceptable amine salt), such as methylamine salt, ethylamine salt, propylamine salt, dimethylamine salt, trimethylamine salt, diethylamine salt, triethylamine salt, tert-butyl amine salt, ethylenediamine salt, hydroxyethylamine salt, dihydroxyethylamine salt, trihydroxyethylamine salt, and amine salt formed with morpholine, piperazine, lysine, respectively.

The term “solvate” refers to a complex with specific proportion formed by the compound of the invention coordinated with a solvent molecule. “Hydrate” refers to a complex formed by the compound of the invention coordinated with water.

The prodrug includes (but is not limited to) carboxylic ester, carbonate, phosphate, nitrate, sulfate, sulfone, sulfoxide, amino compound, carbamate, azo compound, phosphoramide, glucoside, ether, acetal and other forms of the compound.

Preparation Method

The compound of the invention may also optionally be conveniently prepared by combining the various synthetic methods described in this specification or known in the art, such a combination may be easily performed by a skilled person in the art to which the invention belongs.

Preferably, the compound of the invention is carried out as follows:

S1) In an inert solvent (e.g. dioxane), and in the presence of a catalyst (e.g. Pd(dppf)Cl₂) and a base (e.g. K₂CO₃), compound 1 is reacted with compound 2 to obtain compound 3;

S2) in an inert solvent (e.g. DMF), and in the presence of a base (such as Cs₂CO₃), compound 3 is reacted with compound 4 to obtain compound F;

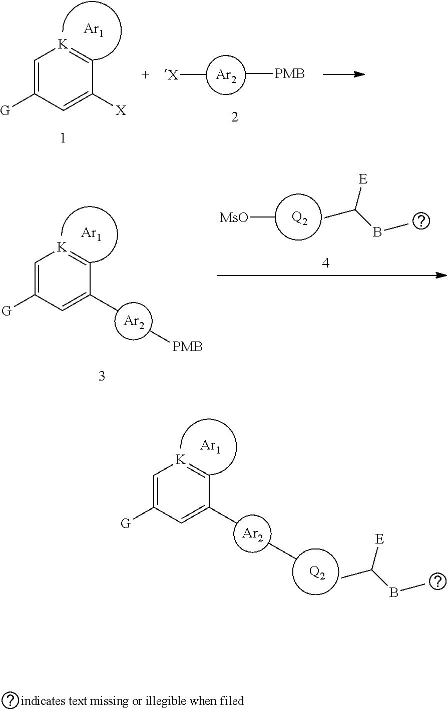

wherein, X and X′ are each independently halogen, OTf or

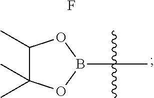

G, K, Ar₁, Ar₂, Q2, E, B, R, and f are defined as above.

Pharmaceutical Composition and Method of Administration

The pharmaceutical compositions of the present invention are used to prevent and/or treat the following diseases: inflammation, cancer, cardiovascular disease, infection, immunological disease, metabolic disease.

The compounds of the present invention can be used in combination with other drugs known to treat or improve similar conditions. When administered in combination, the original administration method and dosage for the drug can remain unchanged, while compound of the present invention may be administered simultaneously or subsequently. Pharmaceutical composition containing one or more known drugs and the compound of the present invention may be preferred when administered in combination with one or more other drugs. The drug combination also includes administering the compound of the present invention and other one or more known drugs at overlapping time. When the compound of the present invention is combined with other one or more drugs, the dosage of the compound of the present invention or known drug may be lower than that of their individual use.

The dosage forms of the pharmaceutical composition of the present invention include (but are not limited to), injection, tablet, capsule, aerosol, suppository, pellicle, pill, liniment for external use, controlled release or sustained-release or nano formulation.

The pharmaceutical composition of the present invention comprises a compound of the present invention or a pharmaceutically acceptable salt and a pharmaceutically acceptable excipient or carrier with safe and effective amount. The “safe and effective amount” refers to the amount of compound is sufficient to significantly improve the condition, not to produce severe side effects. Typically, the pharmaceutical composition contains 1-2000 mg of the compound of the present invention/dosage, and preferrably contains 10-1000 mg of the compound of the present invention/dosage. Preferably, “one dosage” is a capsule or a pill.

“Pharmaceutically acceptable carrier” refers to one or more compatible solid or liquid filler or gel substances, which are suitable for human use, and must be sufficiently pure and of sufficiently low toxicity. “Compatible” herein refers to ability of each component of a composition can be mixed with the compound of the present invention and can be mixed with each other without appreciably reducing the efficacy of the compound. Examples of pharmaceutically acceptable carrier include cellulose and derivatives thereof (such as sodium carboxymethylcellulose, sodium ethylcellulose, cellulose acetate, etc.), gelatin, talc, solid lubricant (such as stearic acid, magnesium stearate), calcium sulfate, vegetable oil (such as soybean oil, sesame oil, peanut oil, olive oil, etc.), polyol (such as propylene glycol, glycerol, mannitol, sorbitol, etc.), emulsifier (such as Tween®), wetting agent (such as lauryl sodium sulfate), colorant, flavoring, stabilizer, antioxidant, preservative, pyrogen-free water, etc.

There is no special limitation of administration mode for the compound or pharmaceutical compositions of the present invention, and the representative administration mode includes (but is not limited to): oral, intratumorally, rectal, parenteral (intravenous, intramuscular or subcutaneous), and topical administration.

Solid dosage forms for oral administration include capsules, tablets, pills, powders and granules. In these solid dosage forms, the active compounds are mixed with at least one conventional inert excipient (or carrier), such as sodium citrate or dicalcium phosphate, or mixed with any of the following components: (a) fillers or compatibilizer, such as starch, lactose, sucrose, glucose, mannitol and silicic acid; (b) binders, such as hydroxymethyl cellulose, alginate, gelatin, polyvinylpyrrolidone, sucrose and arabic gum; (c) humectant, such as, glycerol; (d) disintegrating agent, such as agar, calcium carbonate, potato starch or tapioca starch, alginic acid, certain composite silicates, and sodium carbonate; (e) dissolution-retarding agents, such as paraffin (f) absorption accelerators, such as quaternary ammonium compounds; (g) wetting agents, such as cetyl alcohol and glyceryl monostearate; (h) adsorbents, for example, kaolin; and (i) lubricants such as talc, stearin calcium, magnesium stearate, solid polyethylene glycol, lauryl sodium sulfate, or the mixtures thereof. In capsules, tablets and pills, the dosage forms may also contain buffering agents.

The solid dosage forms such as tablets, sugar pills, capsules, pills and granules can be prepared by using coating and shell materials, such as enteric coatings and any other materials known in the art. They can contain an opaque agent. The release of the active compounds or compounds in the compositions can be released in a delayed mode in a given portion of the digestive tract. Examples of the embedding components include polymers and waxes. If necessary, the active compounds and one or more above excipients can form microcapsules.

Liquid dosage forms for oral administration include pharmaceutically acceptable emulsions, solutions, suspensions, syrups or tinctures. In addition to the active compounds, the liquid dosage forms may contain any conventional inert diluents known in the art such as water or other solvents, solubilizers and emulsifiers, such as ethanol, isopropanol, ethyl carbonate, ethyl acetate, propylene glycol, 1,3-butanediol, dimethyl formamide, as well as oil, in particular, cottonseed oil, peanut oil, corn germ oil, olive oil, castor oil and sesame oil, or the combination thereof.

Besides these inert diluents, the composition may also contain additives such as wetting agents, emulsifiers, and suspending agent, sweetener, flavoring agents and perfume.

In addition to the active compounds, the suspension may contain suspending agent, for example, ethoxylated isooctadecanol, polyoxyethylene sorbitol and sorbitan esters, microcrystalline cellulose, methanol aluminum and agar, or the combination thereof.

The compositions for parenteral injection may comprise physiologically acceptable sterile aqueous or anhydrous solutions, dispersions, suspensions or emulsions, and sterile powders which can be re-dissolved into sterile injectable solutions or dispersions. Suitable aqueous and non-aqueous carriers, diluents, solvents or excipients include water, ethanol, polyols and any suitable mixtures thereof.

The dosage forms for topical administration of compounds of the invention include ointments, powders, patches, aerosol, and inhalants. The active ingredients are mixed with physiologically acceptable carriers and any preservatives, buffers, or propellant if necessary, under sterile conditions.

The treatment method of the present invention can be administered alone or in combination with other treatment means or therapeutic drugs.

When the pharmaceutical compositions are used, a safe and effective amount of compound of the present invention is administrated to a mammal (such as human) in need thereof, wherein the dose of administration is a pharmaceutically effective dose. For a person weighed 60 kg, the daily dose is usually 1-2000 mg, preferably 50-1000 mg. Of course, the particular dose should also depend on various factors, such as the route of administration, patient healthy status, which are well within the skills of an experienced physician.

The present invention also provides a preparation method of pharmaceutical composition comprising the step of mixing a pharmaceutically acceptable carrier with the compound or the pharmacically acceptable salt, stereoisomer, solvate or prodrug thereof of the present invention, thus forming the pharmaceutical composition.

The invention also provides a treatment method comprising the step of administering the compound, or pharmaceutically acceptable salt, stereoisomer, solvate or prodrug thereof of the invention, or administering the pharmaceutical composition of the invention to a subject in need thereof to selectively inhibit RET.

The Invention has the Following Main Advantages:

(1) The compound of the invention has good inhibition ability to RET kinase;

(2) The compound has better pharmacodynamics, pharmacokinetic properties and lower toxic and side effects:

(3) The results show that the compound with both Ar₁ and Ar₂ as five-membered heteroaryl (such as compounds C1 or C2) has better inhibition effect than the compound with Ar₁ and/or Ar₂ as six-membered heteroaryl.

The present invention will be further illustrated below with reference to the specific examples. It should be understood that these examples are only to illustrate the invention but not to limit the scope of the invention. The experimental methods without specific conditions in the following examples usually follow conventional conditions, such as Sambrook et al., Molecular Cloning: Laboratory Manual (New York: Cold Spring Harbor Laboratory Press, 1989), or according to the conditions recommended by the manufacturer. Unless otherwise stated, percentage and parts are calculated by weight.

All major and scientific terms used herein are the same as those familiar to those skilled in the art, unless otherwise defined. In addition, any method or material which is similar or equal to the recorded content may be applied to the method of the present invention. The preferred methods and materials described herein are described only for demonstration purposes.

The compound structure of the present invention was determined by nuclear magnetic resonance (NMR) and Liquid-mass chromatography (LC-MS).

### EXAMPLES

### Example 1 Synthesis of Compound C1

The synthetic route was as follows:

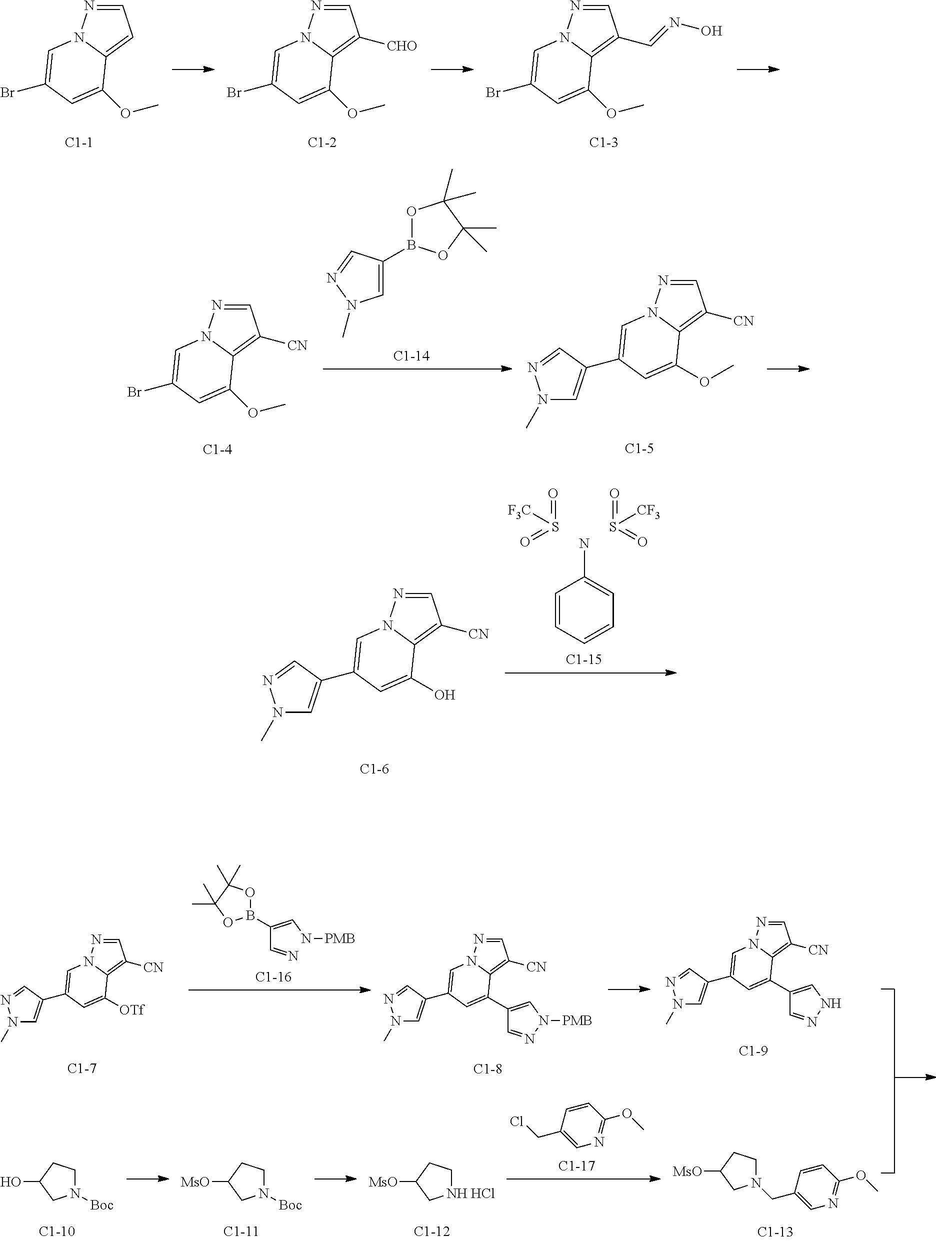

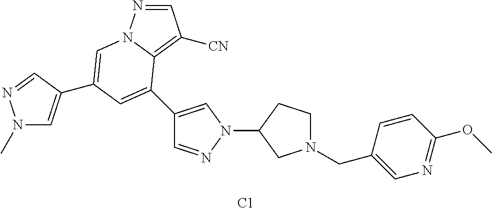

(1) Synthesis of Intermediate C1-9

1. Synthesis of Intermediate C1-2:

C1-1 (15.86 mmol, 3.6 g) and DMF (150 ml) were successively added into a 250 mL single-neck flask, then the reaction solution was cooled to −10° C., and POCl₃ (47.58 mmol, 4.43 ml) was slowly added dropwise. Then they were reacted at −10° C.-5° C. for 1 h, and the temperature was slowly raised to room temperature to react overnight. Water (150 ml) was added slowly for dilution, and the pH was adjusted to 9-10 with 1 N NaOH (350 ml). The reaction solution was filtered, and the filter cake was washed with water, then washed with ether, and the filter cake was dried to obtain 4.0 g of intermediate C1-2.

2. Synthesis of Intermediate C1-3:

C1-2 (15.7 mmol, 4.0 g), HONH₂.HCl (23.5 mmol, 1.64 g) and EtOH/H₂O (16/8 mL) were successively added into a 50 ml single-neck flask, and the mixture was reacted at 50° C. for 4 h. The reaction solution was cooled, concentrated, and the pH was adjusted to >7 with saturated NaHCO₃ aqueous solution. The reaction solution was filtered, and the filter cake was washed with H₂O/Et₂O, and dried to obtain 3.74 g of intermediate C1-3.

3. Synthesis of Intermediate C1-4:

C1-3 (13.8 mmol, 3.74 g) and propionic anhydride (80 mL) were successively added into a 250 mL single-neck flask, and the mixture was reacted at 120° C. overnight. The reaction solution was cooled and evaporated to dryness to obtain 3.43 g of intermediate C1-4.

4. Synthesis of Intermediate C1-5:

C1-4 (13.6 mmol, 3.43 g), C1-14 (16.3 mmol, 3.4 g), Pd (PPh₃)₄ (0.54 mmol, 629 mg), Na₂CO₃ (40.8 mmol, 4.32 g) and Dioxane/H₂O (62/27 mL) were successively added into a 250 mL single-neck flask, and the mixture was reacted at 80° C. overnight under the protection of N₂. LC-MS detection showed the reaction was completed, and the reaction solution was added H₂O for dilution, extracted with EA for three times. The organic phases were dried, evaporated under reduced pressure, purified by silica gel column chromatography to obtain 3.05 g of product (intermediate C1-5).

5. Synthesis of Intermediate C1-6:

C1-5 (12.1 mmol, 3.05 g), DCE (40 mL) and AlCl₃ (42.2 mmol, 5.63 g) were successively added into a 100 mL single-neck flask, and the mixture was reacted at 80° C. overnight under the protection of N₂. DCE was added to dilute and H₂O was added for quenching, the reaction was stirred under r.t. for 3 h. The reaction solution was evaporated to dryness under reduced pressure, slurried with methanol, filtered. The filtrate was concentrated, then slurried with water, filtered and the filter cake was dried to obtain 2.93 g of intermediate C1-6.

6. Synthesis of Intermediate C1-7:

C1-6 (11.57 mmol, 2.93 g), DMA (35 mL), DIPEA (23.14 mmol, 2.99 g) and N-Phenyl-bis(trifluoromethanesulfonimide)(12.72 mmol, 4.54 g) were successively added into a 100 mL single-neck flask, and the mixture was reacted at r.t. overnight under the protection of N₂. H₂O was added for dilution, and the reaction was stirred under r.t. for 30 min. The reaction solution was filtered. The filter cake was dissolved in DCM. The organic phase was dried, concentrated, and purified by silica gel column chromatography to obtain 2.4 g of intermediate C1-7.

7. Synthesis of Compound C1-8:

C1-7 (0.6 mmol, 223 mg), C1-16 (1.2 mmol, 374 mg), Pd (dppf) Cl₂ (0.06 mmol, 50 mg), K₂CO₃ (0.9 mmol, 124 mg) and Dioxane (10 mL) were successively added into a 25 mL single-neck flask, and the mixture was reacted at 90° C. overnight under the protection of N₂. LC-MS detection showed the reaction was completed, and the reaction solution was added H₂O for dilution, extracted with EA for three times. The organic phases were dried, evaporated under reduced pressure, purified by silica gel column chromatography to obtain 138 mg of product (intermediate C1-8).

8. Synthesis of Compound C1-9:

C1-8 (0.52 mmol, 210 mg) and TFA (4 ml) were successively added into a 25 mL single-neck flask, and the mixture was reacted at 70° C. for 4 h. LCMS detection showed the reaction was completed. The reaction solution was evaporated to dryness under reduced pressure to obtain 180 mg yellow solid which was directly used for the next reaction.

9. Synthesis of Intermediate C1-13:

C1-10 (10 mmol, 1.87 g), DCM (20 ml), DIPEA (30 mmol, 3.87 g) and MsCl (10 mmol, 1.15 g) were successively added into a 100 mL single-neck flask, and the mixture was reacted at r.t for 4 h. TLC detection showed the reaction was completed. The reaction solution was diluted with DCM, washed with H₂O for three times, the organic phases were dried, and evaporated under reduced pressure to obtain 2.55 g of intermediate C1-13, which was directly used for the next reaction.

The above product was dissolved in 10 mL DCM, HCl/dioxane (4 M, 20 mL) was added into it, and stirred at room temperature for 4 h. The reaction solution was diluted with DCM, filtered, the filtrate cake was washed with ether and dried to obtain 1.65 g of crude white solid, which was directly used for the next reaction.

C1-12 (3.2 mmol, 642 mg), DMF (10 mL), DIPEA (32 mmol, 4.13 g) and C1-17 (3.5 mmol, 552 mg) were successively added into a 25 mL single-neck flask, and the mixture was reacted at r.t for 4 h. TLC detection showed the reaction was completed, and the reaction solution was added H₂O for dilution, extracted with EA for three times. The organic phases were dried, evaporated under reduced pressure, purified by silica gel column chromatography to obtain 126 mg of product.

10. Synthesis of Compound C1:

C1-9 (0.2 mmol, 56 mg), C1-13 (0.22 mmol, 64 mg), Cs₂CO₃ (0.4 mmol, 132 mg) and DMF (2 mL) were successively added into a 25 mL single-neck flask, and the mixture was reacted at 80° C. for 5 h. LC-MS detection showed the reaction was completed, and the reaction solution was added H₂O for dilution, extracted with EA for three times. The organic phases were dried, evaporated under reduced pressure, and purified by silica gel column chromatography to obtain 34 mg of product as a yellow solid with a purity of 97.7%. [M+H]: 480.2. 1H NMR (400 MHz, DMSO-d6) δ9.19 (d, J=1.5 Hz, 1H), 8.66 (s, 1H), 8.38 (s, 1H), 8.34-8.27 (m, 1H), 8.11 (dd, J=7.9, 1.6 Hz, 2H), 7.87 (d, J=0.8 Hz, 1H), 7.83 (d, J=1.5 Hz, 1H), 7.67 (dd, J=8.5, 2.4 Hz, 1H), 6.76 (d, J=8.5 Hz, 1H), 4.99 (s, 1H), 3.89 (s, 3H), 3.82 (s, 3H), 3.68-3.53 (m, 2H), 2.89 (dt, J=46.2, 8.6 Hz, 3H), 2.57 (d, J=7.3 Hz, 1H), 2.48-2.33 (m, 1H), 2.25-2.11 (m, 1H).

### Example 2 Synthesis of Compound C2

The synthetic route was as follows:

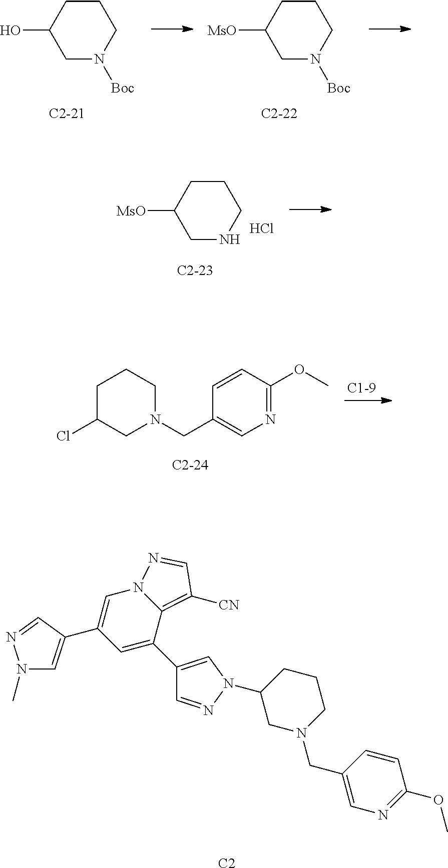

1. Synthesis of Intermediate C2-24:

C2-21 (10 mmol, 2.01 g), DCM (20 ml), DIPEA (30 mmol, 3.87 g) and MsCl (15 mmol, 1.72 g) were successively added into a 100 mL single-neck flask, and the mixture was reacted at r.t for 4 h. TLC detection showed the reaction was completed. The reaction solution was diluted with DCM, washed with H₂O for three times, the organic phase was dried, and evaporated under reduced pressure to obtain 2.8 g of intermediate C2-24 as a brown oil, which was directly used for the next reaction.

The above products were dissolved in 10 mL DCM, HCl/dioxane (4 M, 20 mL) was added into it, stirred at room temperature for 4 h, diluted with DCM, filtered, the filtrate cake was washed with ether, dried to obtain 2.0 g of crude product as a brown solid, which was directly used for the next reaction.

C2-23 (1 mmol, 179 mg), DMF (2 mL), DIPEA (3 mmol, 387 mg) and 5-chloromethyl-2-methoxypyridine (1.2 mmol, 188 mg) were successively added into a 25 mL single-neck flask, and the mixture was reacted at r.t for 6 h. TLC detection showed the reaction was completed, and the reaction solution was added H₂O for dilution, extracted with EA for three times. The organic phases were dried, evaporated under reduced pressure, purified by silica gel column chromatography to obtain 482 mg of compound C2-2 as a yellow oil.

2. Synthesis of Compound C2

C1-9 (0.55 mmol, 106 mg), C2-24 (0.6 mmol, 97 mg), Cs₂CO₃ (1.1 mmol, 239 mg) and DMF (5 mL) were successively added into a 25 mL single-neck flask, and the mixture was reacted at 80° C. for 5 h. LC-MS detection showed the reaction was completed, and the reaction solution was added H₂O for dilution, extracted with EA for three times. The organic phases were dried, evaporated under reduced pressure, and purified by silica gel column chromatography to obtain 12 mg of product as a yellow solid with a purity of 91.7%. [M+H]: 494.2.

### Example 3 Synthesis of Compound C3

The synthetic route was as follows:

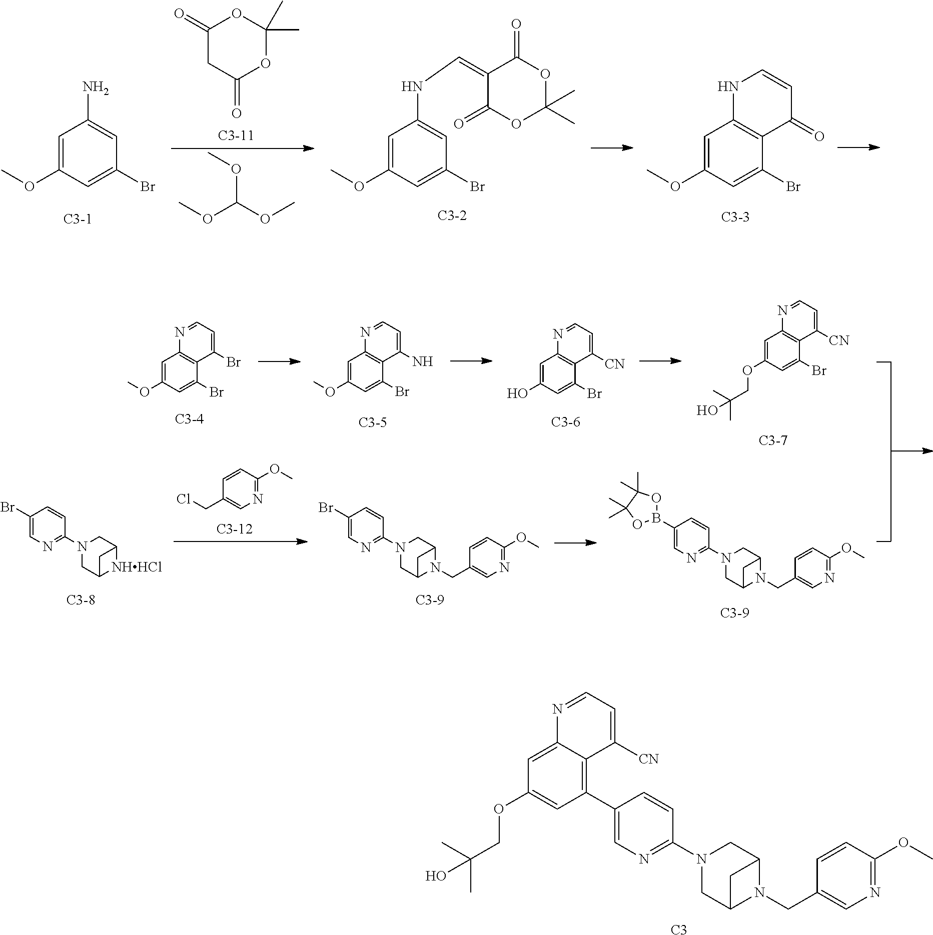

1. Synthesis of Intermediate C3-7

C3-11 (20 mmol, 2.9 g) and trimethyl orthoformate (30 mL) were successively added into a 100 mL three-neck flask, and the mixture was reacted at 100° C. for 2 hours under the protection of argon. The raw material C3-1 (20 mmol, 4 g) was dissolved in trimethyl orthoformate (30 mL). The above solution was slowly added dropwise to the reaction, after addition, the reaction solution was reacted at 100° C. overnight under stirring. The reaction solution was concentrated, and the crude product was purified by silica gel column chromatography (PE/EA=10:1-3:1) to obtain 4.16 g of intermediate C3-2.

C3-2 (10 mmol, 4.16 g) and diphenyl ether (30 mL) were successively added into a 50 mL three-neck flask, and the mixture was reacted at 260° C. for 15 minutes under the protection of argon. The reaction solution was cooled to room temperature, and 30 mL petroleum ether was added. The stock was filtrated. The filter cake was purified by silica gel column chromatography (first PE/EA=1:1, then EA/MeOH=50:1) to obtain 750 mg of intermediate C3-3.

C3-3 (3 mmol, 750 mg) and DMF (10 mL) were successively added into a 50 mL three-neck flask, and the mixture was reacted under ice bath. The reaction solution was cooled to 0° C., PBr₃ (2.5 mL) was added dropwise under the protection of argon. After addition, the reaction was stirred at room temperature for 2 hours. TLC showed that the reaction was completed. Water (10 mL) and ethyl acetate (20 mL) were added into it, and the aqueous phase was adjusted to pH=7-8 with saturated NaHCO₃, extracted with ethyl acetate for three times. The organic phases were combined, dried and concentrated to obtain crude product, which was purified by silica gel column chromatography (PE/EA=10:1) to obtain about 700 mg of intermediate C3-4.

C3-4 (4 mmol, 680 mg), zinc cyanide (4 mmol, 470 mg), Pd (PPh₃)₄ (0.4 mmol, 450 mg) and DMF (10 mL) were successively added into a 50 mL single-neck flask, after replacing argon, and the mixture was reacted at 90° C. overnight under the protection of argon. The reaction solution was cooled to room temperature. The mother liquor was filtered, water (10 mL) and ethyl acetate (20 mL) were added into it, and the aqueous phase was extracted with ethyl acetate for three times. The organic phases were combined, dried and concentrated to obtain a crude product, which was purified by silica gel column chromatography (PE/EA=20:1-10:1) to obtain about 300 mg of intermediate C3-5.

C3-5 (0.76 mmol, 200 mg), AlCl₃ (2.3 mmol, 300 mg) and toluene (10 mL) were successively added into a 10 mL reaction tube, and the mixture was reacted at 110° C. for 48 hours. The pH was adjusted to 5-6 with 10% NaOH. The aqueous phase was extracted with ethyl acetate. The organic phases were combined, washed with saturated salt water, dried and concentrated, and purified by silica gel column chromatography (PE/EA=5:1-2:1) to obtain 100 mg of intermediate C3-6.

C3-6 (0.4 mmol, 100 mg), dimethyl ethylene oxide (4 mmol, 300 mg), K₂CO₃ (0.21 mmol, 167 mg) and DMF (5 mL) were successively added into a 10 mL reaction tube, and the tube was sealed to react at 95° C. for 16 h. The reaction solution was cooled to room temperature, and water and ethyl acetate were added. The aqueous phase was extracted with ethyl acetate, and the organic phases were combined, washed with saturated salt water, dried and concentrated, and purified by silica gel column chromatography (PE/EA=5:1-1:10) to obtain 80 mg of intermediate C3-7.

2. Synthesis of Intermediate C3-10

C3-8 (1.0 mmol, 290 mg). C3-12 (1.1 mmol, 179 mg), DIPEA (8 mmol, 1 g) and DMF (5 mL) were successively added into a 10 mL reaction tube, and the mixture was reacted at room temperature for 16 h under the protection of argon. Water and ethyl acetate were added into it. The aqueous phase was extracted with ethyl acetate, and the organic phases were combined, washed with saturated salt water, dried and concentrated, and purified by silica gel column chromatography (PE/EA=1:1-1:10) to obtain 140 mg of intermediate C3-9.

C3-9 (0.4 mmol, 140 mg), bis(pinacolato)diboron (0.5 mmol, 130 mg), KOAc (0.56 mmol, 55 mg), Pd (dppf) Cl₂ (0.04 mmol, 27 mg) and dioxane (3 mL) were successively added into a 10 mL reaction tube, after being replaced by argon, and the mixture was reacted at 100° C. overnight under the protection of argon. The reaction solution was cooled to room temperature. The mother liquor was filtered. The filtrate was dried and concentrated, and purified by silica gel column chromatography (EA/MeOH=50:1) to obtain 120 mg of intermediate C3-10.

3. Synthesis of Compound C3

C3-7 (0.3 mmol, 96 mg), C3-10 (0.3 mmol, 125 mg), K₂CO₃ (0.9 mmol, 100 mg), Pd(dppf)Cl₂ (0.03 mmol, 25 mg) and dioxane (3 mL) were successively added into a 10 mL reaction tube, after being replaced by argon, and the mixture was reacted at 90° C. overnight under the protection of argon. The reaction solution was cooled to room temperature. The mother liquor was filtered. The filtrate was concentrated, water and ethyl acetate were added. The aqueous phase was extracted with ethyl acetate, and the organic phases were combined, washed with saturated salt water, dried and concentrated. The crude product was separated by liquid phase preparation to obtain 30 mg of Compound C3.

¹H NMR (400 MHz, Chloroform-d) δ 8.94 (d, J=4.4 Hz, 1H), 8.11 (d, J=2.3 Hz, 1H), 7.67-7.58 (m, 2H), 7.55 (dd, J=8.1, 2.5 Hz, 2H), 7.32 (d, J=2.7 Hz, 1H), 6.69 (dd, J=19.1, 8.6 Hz, 2H), 4.12 (q, J=7.1 Hz, 2H), 4.02 (s, 3H), 3.92 (s, 2H), 3.87-3.79 (m, 2H), 3.67-3.60 (m, 3H), 3.49 (s, 2H), 2.70 (m, 1H), 1.42 (s, 6H).

### Example 4 Synthesis of Compound C4

The synthetic route was as follows:

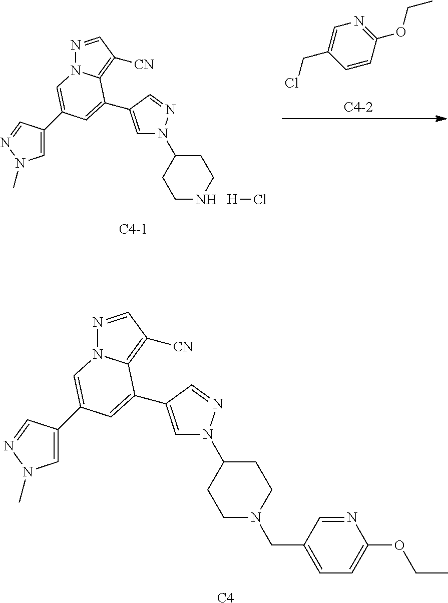

Synthesis of Compound C4:

Compound C4-1 (145 mg, 0.34 mmol) and compound C4-2 (117 mg, 0.69 mmol) were dissolved in 10 mL DMF, then 0.3 mL triethylamine was added into it, and stirred at room temperature overnight under the protection of argon. The reaction solution was concentrated, diluted with water, extracted with dichloromethane. The combined organic phases were washed with saturated salt water, dried over anhydrous sodium sulfate, concentrated to obtain a crude product, then it was slurried with MeOH and filtered to obtain 30 mg of the target compound. MS [M+H] 520.1.

1H NMR (400 MHz, DMSO-d₆) δ 10.89 (br, 1H), 9.26 (s, 1H), 8.66 (s, 1H), 8.45-8.33 (t, 3H), 8.12 (s, 1H), 7.94-7.92 (t, 2H), 7.79-7.78 (d, 1H, J=1.27 Hz), 7.11-7.08 (d, 1H, J=8.54 Hz), 6.92-6.90 (d, 1H, J=8.84 Hz), 4.54-4.51 (d, 2H, J=14.74 Hz), 4.36-4.31 (q, 3H), 3.89 (s, 3H), 3.47-3.44 (m, 2H), 3.33 (s, 3H), 3.12-3.09 (m, 2H), 1.35-1.32 (t, 3H).

### Example 5 Synthesis of Compound C5

The synthetic route was as follows:

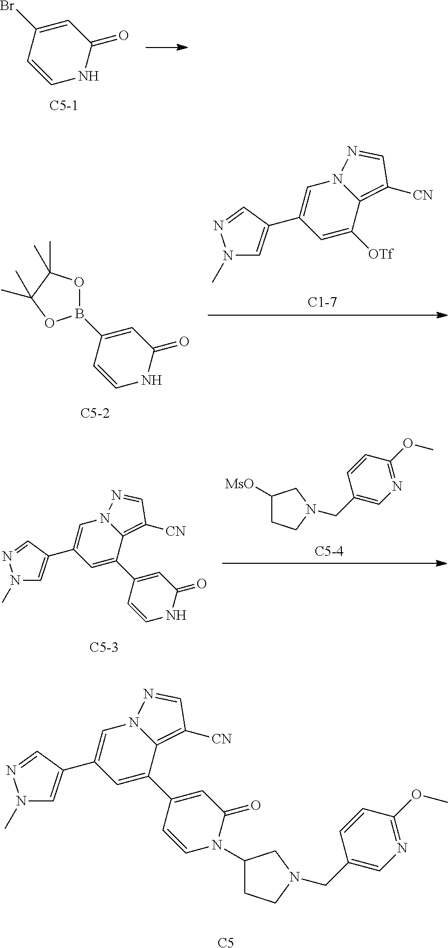

Synthesis of Intermediate C5-2:

C5-1 (5 mmol, 870 mg), bis(pinacolato)diboron (5.25 mmol, 1.33 g). KOAc (7 mmol, 691 mg), Pd (dppf) Cl₂ (0.5 mmol, 400 mg) and dioxane (10 mL) were successively added into a 50 mL single-neck flask, after being replaced by argon, and the mixture was reacted at 90° C. overnight under the protection of argon. The reaction solution was cooled to room temperature. The mother liquor was filtered. The filtrate was dried and concentrated, and purified by silica gel column chromatography (DCM/MeOH=50:1) to obtain 880 mg of compound C5-2.

Synthesis of Intermediate C5-3:

C5-2 (0.4 mmol, 88 mg), C1-7 (0.2 mmol, 75 mg), K₂CO₃ (0.3 mmol, 42 mg), Pd(dppf)Cl₂ (0.034 mmol, 25 mg) and dioxane (5 mL) were successively added into a 50 mL single-neck flask, after being replaced by argon, and the mixture was reacted at 60° C. overnight under the protection of argon. The reaction solution was cooled to room temperature. The mother liquor was filtered. The filtrate was concentrated, and water and ethyl acetate were added. The aqueous phase was extracted with ethyl acetate, and the organic phases were combined, washed with saturated salt water, dried and concentrated, and purified by silica gel column chromatography (DCM/MeOH=50:1) to obtain 50 mg of compound C5-3.

Synthesis of Compound C5:

C5-3 (0.13 mmol, 40 mg), C5-4 (0.19 mmol, 54 mg), K₂CO₃ (0.21 mmol, 30 mg) and DMF (2 mL) were successively added into a 10 mL reaction tube, and the mixture was reacted at 90° C. overnight under the protection of argon. The reaction solution was cooled to room temperature, and water and ethyl acetate were added. The aqueous phase was extracted with ethyl acetate, and the organic phases were combined, washed with saturated salt water, dried and concentrated, and purified by silica gel column chromatography (DCM/MeOH=20:1) to obtain 10 mg of Compound C5.

¹H NMR (400 MHz, Chloroform-d) δ 8.71 (d, J=1.5 Hz, 1H), 8.33 (d, J=5.4 Hz, 1H), 8.09 (d, J=2.3 Hz, 1H), 7.80 (dd. J=6.9, 0.8 Hz, 1H), 7.72-7.69 (m, 2H), 7.50 (dd, J=5.9, 1.5 Hz, 1H), 7.10 (dd, J=5.2, 1.5 Hz, 1H), 6.94 (d, J=1.4 Hz, 1H), 6.79-6.58 (m, 2H), 4.20-4.06 (m, 1H), 4.00 (s, 3H), 3.90 (s, 3H), 3.18 (s, 2H), 2.59-2.21 (m, 2H), 2.06 (d, J=8.8 Hz, 2H), 1.80 (m, 2H).

### Example 6 Synthesis of Compound C6

The synthetic route was as follows:

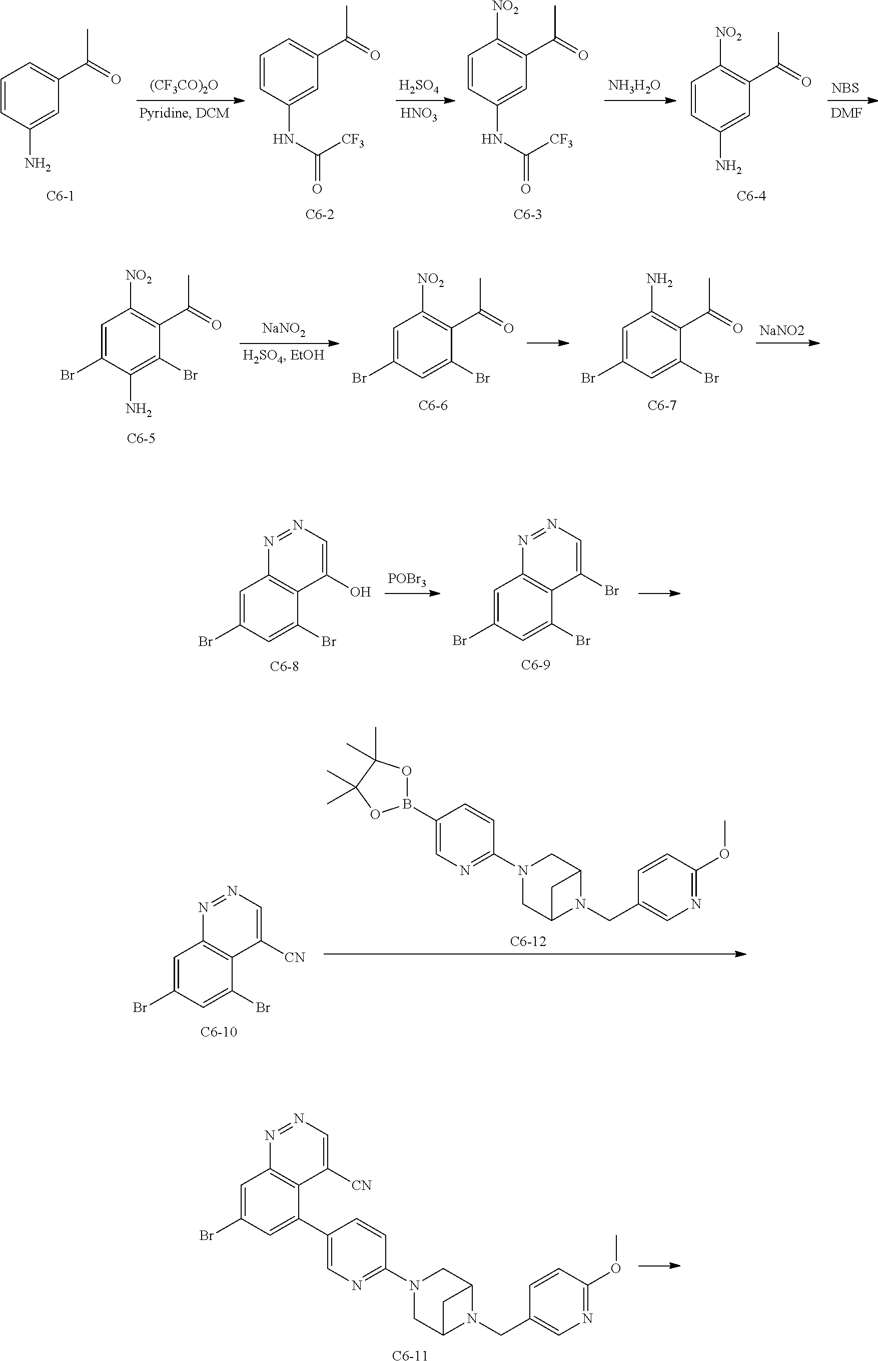

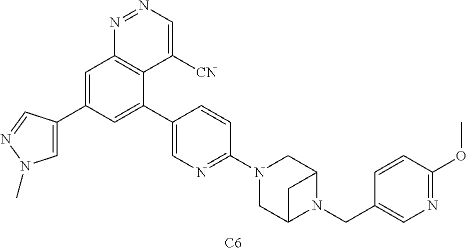 [SMILES: COc1ccc(CN2C3CC2CN(c2ccc(-c4cc(-c5cnn(C)c5)cc5nncc(C#N)c45)cn2)C3)cn1 ; "C6"]

Synthesis of Intermediate C6-2:

C6-1 (2.5 g, 18.5 mmol) and pyridine (1.6 mL) were dissolved in dichloromethane (15 mL), trifluoroacetic anhydride (4.08 g, 19.42 mmol) was added into it dropwise under ice bath, and the mixture was naturally raised to room temperature and stirred overnight. The reaction solution was added to water, extracted with dichloromethane, dried and concentrated, and purified by column chromatography to obtain 4.03 g of compound C6-2.

Synthesis of Intermediate C6-3:

C6-2 (3.43 g, 14.8 mmol) was dissolved in concentrated sulfuric acid (30 mL), concentrated nitric acid (65%, 1.73 mL) was slowly added into it dropwise at −20° C. for over 10 minutes, and the mixture was stirred at this temperature for 1 hour. TLC showed that the reaction was almost completed. The reaction solution was poured into an ice water bath, extracted with ethyl acetate. The organic phases were combined, washed with saturated sodium bicarbonate solution and salt water successively, the organic phase was dried, concentrated, the residue was slurried twice with petroleum ether, and filtered to obtain 2.9 g of compound C6-3.

Synthesis of Intermediate C6-4:

C6-3 (2.9 g) was added to ammonia (100 mL), and the mixture was stirred at 50° C. for 1 hour. TLC showed that the reaction was completed. The reaction solution was cooled, filtered. The solid was washed with water and dried to obtain 1.63 g of compound C6-4.

Synthesis of Intermediate C6-5:

C6-4 (1.56 g, 8.68 mmol) was dissolved in DMF (30 mL), NBS (3.4 g, 19.1 mmol) was added into it at 0° C., and the mixture was stirred at room temperature for 15 hours. The reaction solution was poured into water, extracted with methyl tert-butyl ether, dried, concentrated and purified by column chromatography to obtain 1.76 g of compound C6-5.

Synthesis of Intermediate C6-6:

C6-5 (1.76 g, 5.2 mmol) was added to ethanol (25 mL) and concentrated sulfuric acid (3.22 g), sodium nitrite (1.8 g, 26 mmol) was added into it at 60° C., the mixture was slowly raised to 90° C., and the reaction solution was stirred at 90° C. for 2 hours. TLC showed that the reaction was completed. The reaction solution was cooled, poured into ice water, and filtered. The filter cake was washed with water, and dried to obtain 1.55 g of compound C6-6.

Synthesis of Intermediate C6-7:

Compound C6-6 (1.5 g, 0.2 mmol) was dissolved in acetic acid (23 ml), iron powder (1.56 g, 6 mmol) was added in batches at 90° C., and the mixture was reacted at 90° C. for 1 hour. TLC showed that the reaction was completed. The reaction solution was cooled, and the solvent was removed under reduced pressure. The residue was added ethyl acetate, filtered, and the filter cake was washed with ethyl acetate. The organic phases were combined, washed with saturated sodium bicarbonate, washed with salt water, dried, and concentrated, and purified by column chromatography to obtain 0.79 g of compound C6-7.

Synthesis of Compound C6-8:

Compound C6-7 (293 mg) was added into hydrochloric acid (6M, 3.6 mL), and sodium nitrite aqueous solution (75.9 mg, 0.4 mL) was added into it dropwise at −10° C.˜0° C. After addition, the reaction solution was stirred at 0° C. for 1.5 hours, and the mixture was raised to room temperature naturally and stirred overnight. The reaction solution was stirred at 85° C. for 4.5 hours, cooled, filtered, and the filter cake was washed with water and ether successively, and dried in vacuum to obtain 153 mg of compound C6-8.

¹H NMR (400 MHz, DMSO-d) δ 13.43 (br, 1H), 7.77 (d, J=1.68 Hz, 1H), 7.71-7.70 (m, 2H).

Synthesis of Compound C6-10:

C6-8 (575 mg, 1.89 mmol) was added into chloroform (35 mL) and phosphorus oxybromide (10.78 g) was added into it, and the mixture was reacted at 70° C. overnight under stirring. The reaction solution was cooled, concentrated. Ice water was added into the residue, and ethyl acetate was added for extraction twice. The organic phases were combined, washed with saturated sodium bicarbonate and salt water successively, fully dried, and concentrated to obtain 534 mg of crude product C6-9. 512 mg of crude product compound C6-9 was added into 10 mL DMSO, and cuprous cyanide (150 mg) was added, and the mixture was reacted at 100° C. for 5.5 hours under the protection of nitrogen. TLC detection showed that the reaction was completed. The reaction solution was cooled, and ethyl acetate (100 mL) was added into it, and filtered. The filter cake was added ammonia water, and extracted with ethyl acetate. The organic phases were combined, washed with salt water, dried, concentrated, and purified by column chromatography to obtain 90 mg of compound C6-10.

¹H NMR (400 MHz, DMSO-d) δ 9.5999 (s, 1H), 8.88 (d, J=1.92 Hz, 1H), 8.32 (d, J=1.88 Hz, 1H).

Synthesis of Compound C6-11:

Compound C6-10 (152 mg, 0.48 mmol), compound C6-12 (185 mg, 0.44 mmol), potassium carbonate (132 mg, 0.96 mmol) and [1,1′-bis (diphenylphosphine) ferrocene]palladium dichloride dichloromethane complex (26.3 mg, 0.36 mmol) were added to a mixture of DMF (4 mL) and water (0.4 mL), and the reaction solution was reacted at room temperature overnight under the protection of argon. The reaction solution was added water and extracted with ethyl acetate. The organic phases were dried and concentrated, and purified by column chromatography to obtain 25 mg of compound C6-11.

Synthesis of Compound C6:

Compound C6-11 (15 mg), 1-methylpyrazole-4-boronic acid pinacol ester (12 mg), [1,1′-bis (diphenylphosphine) ferrocene] palladium dichloride dichloromethane complex (3 mg) and potassium carbonate (8.28 mg) were added into a mixture of dioxane (1.8 mL) and water (0.18 mL), and the mixture was stirred overnight at room temperature under the protection of argon. The reaction solution was added ethyl acetate for extraction, dried and concentrated, and purified by column chromatography to obtain Compound C6.

### Example 7 Synthesis of Compound C7

The synthetic route was as follows:

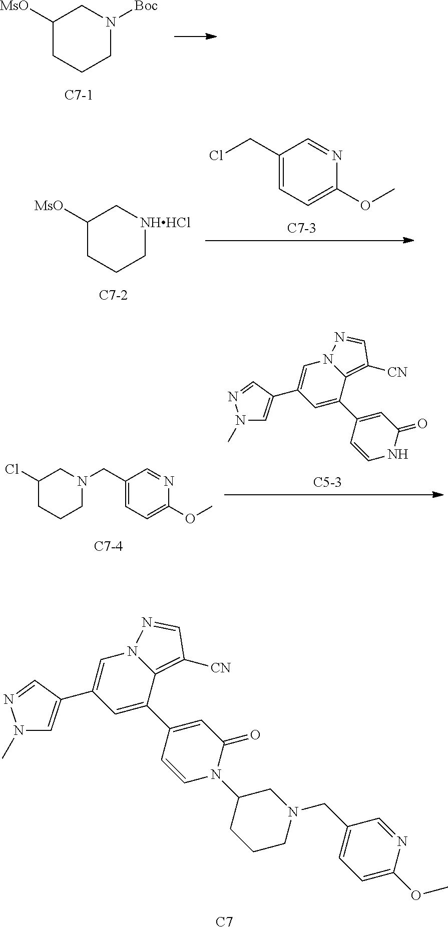

Synthesis of Intermediate C7-2

C7-1 (1.79 mmol, 500 mg) and 15 mL hydrochloric acid/dioxane (4 M) were added into a 50 mL single-neck flask, and the mixture was reacted at room temperature overnight. TLC showed that the reaction was completed, and the reaction solution was concentrated to obtain 400 mg of crude product which was directly followed by the next step.

Synthesis of Intermediate C7-4

C7-2 (1.08 mmol, 230 mg), C7-3 (1.1 mmol, 179 mg), DIPEA (8 mmol, 1 g) and DMF (5 mL) were successively added into a 10 mL reaction tube, and the mixture was reacted at 60° C. for 4 h under the protection of argon. Water and ethyl acetate were added into it. The aqueous phase was extracted with ethyl acetate, and the organic phases were combined, washed with saturated salt water, dried and concentrated, and purified by silica gel column chromatography (PE/EA=1:10) to obtain 100 mg of product as a brown oil.

Synthesis of Compound C7

C7-4 (0.175 mmol, 55 mg), C5-3 (0.19 mmol, 46 mg), K₂CO₃ (0.26 mmol, 36 g) and DMF (1 mL) were successively added into a 10 mL reaction tube, and the mixture was reacted at 80° C. for 16 h under the protection of argon. The reaction solution was cooled to room temperature, water and ethyl acetate were added. The aqueous phase was extracted with ethyl acetate, and the organic phases were combined, washed with saturated salt water, dried and concentrated, and purified by silica gel column chromatography (EA/MeOH=15:1) to obtain 12 mg of Compound C7.

1H NMR (400 MHz, Methanol-d4) δ 9.06 (d, J=1.4 Hz, 1H), 8.42 (s, 1H), 8.30 (d, J=5.3 Hz, 1H), 8.25-8.11 (m, 2H), 8.00 (s, 1H), 7.87-7.70 (m, 2H), 7.25 (dd, J=5.2, 1.4 Hz, 1H), 7.06 (d, J=1.3 Hz, 1H), 6.80 (d, J=8.5 Hz, 1H), 4.41 (d, J=13.2 Hz, 1H), 3.96 (s, 3H), 3.84 (s, 3H), 3.61 (s, 2H) 3.72-3.51 (m, 1H), 3.28-3.10 (m, 2H), 2.92 (q, J=8.3 Hz, 1H), 2.39-2.12 (m, 2H), 2.00-1.74 (m, 2H).

### Example 8 Synthesis of Compound C8

The synthetic route was as follows:

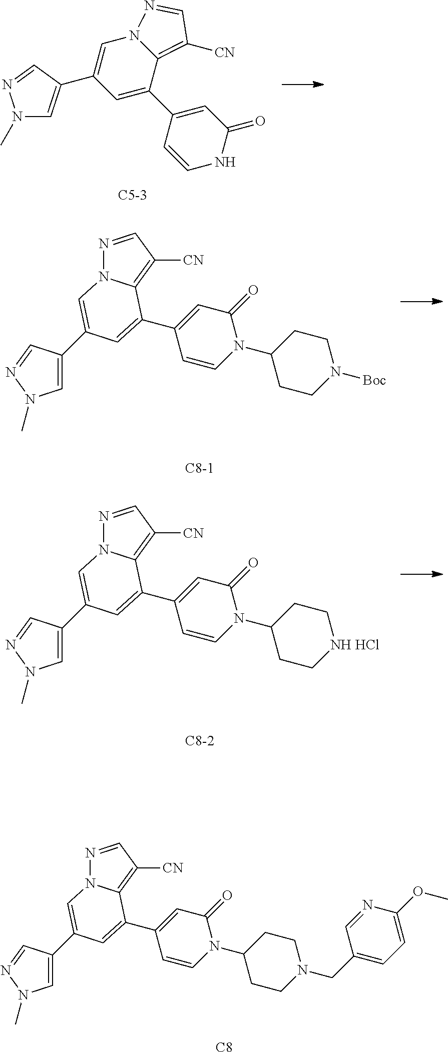

Synthesis of Intermediate C8-1

C5-3 (316 mg, 1 mmol), N-Boc-4-chloropiperidine (263 mg, 1.2 mmol) and N, N-dimethylformamide (6 mL) were added into a 50 mL single-neck flask, and potassium carbonate (414 mg, 3 mmol) was added under stirring, then the mixture was reacted at 80° C. overnight. The reaction solution was cooled to room temperature, diluted with water (60 mL), extracted with ethyl acetate (3×10 mL). The organic phases were combined, washed with saturated salt water (2×10 mL), dried over anhydrous sodium sulfate, filtered, concentrated and purified by column chromatography to obtain 199 mg of product.

Synthesis of Intermediate C8-2

C8-1 (100 mg, 0.2 mmol) and 1,4-dioxane (5 mL) were added into a 50 mL single-neck flask, and HCl/dioxane (3 mL) was added dropwise, the mixture was reacted at room temperature for 2 hours. Solids were precipitated, filtered and dried to obtain 50 mg of product.

Synthesis of Compound C8

C8-2 (50 mg, 0.115 mmol), 5-(chloromethyl)-2-methoxypyridine (36 mg, 0.23 mmol) and N, N-dimethylformamide (5 mL) were added into a 50 mL single-neck flask, and triethylamine (116 mg, 1.15 mmol) was added under stirring, then the mixture was reacted at room temperature overnight. The reaction solution was diluted with water (50 mL), extracted with ethyl acetate (3×10 mL). The organic phases were combined, washed with saturated salt water (2×10 mL), dried over anhydrous sodium sulfate, filtered, concentrated and purified by column chromatography to obtain 27.1 mg of Compound C8 with a purity of 99.2%.

1H NMR (400 MHz, CDCl₃) δ 8.71 (d, J=1.2 Hz, 1H), 8.31-8.29 (m, 2H), 7.80-7.71 (m, 3H), 7.50 (d, J=0.88, 1H), 7.06 (dd, J=5.16, 1.28, 1H), 6.92 (s, 1H), 6.75 (d, J=8.48, 1H), 5.25 (br, 1H), 4.00 (s, 3H), 3.93 (s, 3H), 3.61 (s, 2H), 2.87 (br, 2H), 2.70-2.30 (m, 2H), 2.20 (br, 2H), 1.98 (br, 2H).

### Example 4 Bioactivity Test

The bioactivity test for the compounds of the present invention was carried out.

The experimental process of biological activity test was as follows:

The activity of the compounds prepared in Examples against wild-type RET kinase was screened using the Kinase activity Assay method at ATP Km concentration, and staurosporine was used as a reference substance. The biological activity screening of the compounds will be determined repeatedly at 10 concentrations.

1. Sample to be Tested

Each sample was prepared into a solution with a concentration of 10 mM.

2. Experimental Method

(1) Preparing Basic Buffer Solution and Quenching Buffer Solution for Experimental Kinase

20 mM Hepes (pH 7.5), 10 mM MgCl₂, 1 mM EGTA, 0.02% Brij35, 0.02 mg/ml BSA, 0.1 mM Na₃VO₄, 2 mM DTT, 1% DMSO.

(2) Preparing Compounds for Experimental Kinase

The test compound was dissolved in 100% dimethyl sulfoxide to a specific concentration. Integra Viaflo Assist was used to assist DMSO for (continuous) dilution.

(3) Reaction Steps:

Adding kinase into the newly prepared basic reaction buffer:

Adding any desired cofactors to the substrate solution;

Adding wild-type RET kinase into the substrate solution and gently mixing;

Using Acoustic technology (Echo 550; nanoliter range) to feed the compounds in 100% of dimethyl sulfoxide into the kinase reaction mixture and incubated at room temperature for 20 minutes.

Adding 33P-ATP (specific activity 10 Ci/1) into the reaction mixture to start the reaction.

Incubated at room temperature for 2 hours;

Radioactivity was detected by filter-binding method;

Kinase activity data was expressed as the percentage of remaining kinase activity in the test sample compared to the vehicle (dimethyl sulfoxide) reaction. Prism (GRAPHPAD software) was used to obtain IC50 value and curve-fitting.

The obtained inhibitory activity IC50 (nM) values of the samples against wild-type RET were shown in Table 1.

TABLE 1

| Compound | Wild-RET IC50 (nM) |
|---|---|
| Staurosporine | 2.07 |
| C1 | 1.56 |
| C2 | 3.52 |
| C3 | 226 |
| C4 | 50.7 |
| C5 | 40.7 |
| C7 | 27.3 |
| C8 | 58 |

It can be seen from the above table that the compounds synthesized in the present application have good inhibitory ability against wild-type RET kinase through in vitro biological activity screening compared to the reference substance Staurosporine. In addition, the research of the present invention shows that, when other groups are the same, the compound with both Ar₁ and Ar₂ as five-membered heteroaryl (such as compounds C1 or C2) has better inhibition effect than the compound with Ar₁ and/or Ar₂ as six-membered heteroaryl, which is expected to be further developed into drugs for regulating RET kinase activity or treating RET-related diseases.

The above description is only examples of the present invention, and does not limit the patent scope of the present invention. Any equivalent modifications made by using the contents of the present specification or directly or indirectly applied to other related technical fields are included in the scope of the present invention.

1. A compound of Formula F, or a pharmaceutically acceptable salt, stereoisomer, solvate or prodrug thereof, 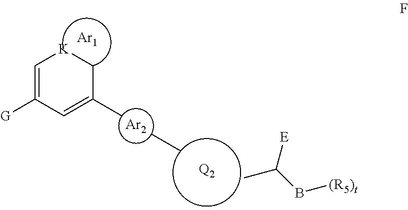
wherein,
G is selected from A-Z₁— or D;
Ar₁ is a substituted or unsubstituted 5-6 membered heteroaryl containing 1-4 N atoms, wherein the “substituted” means being substituted by one or more groups selected from the group consisting of H, CN, halogen, methyl, ethyl and cyclopropyl;
Ar₂ is selected from the substituted or unsubstituted group consisting of 5-6 membered aryl and 5-6 membered heteroaryl, wherein the “substituted” means being substituted by one or more groups selected from the group consisting of C1-C6 alkyl, halogen, hydroxyl, oxo (═O), C1-C6 heteroalkyl, C1-C6 alkoxy, C3-C14 cycloalkyl, 3-14 membered heterocycloalkyl and cyano;
K is selected from C or N;
Q₂ is selected from the group consisting of saturated 4-7 membered monocyclic heterocyclyl, saturated 7-8 membered bridged heterocycle, saturated 7-11 membered spiro heterocyclyl, 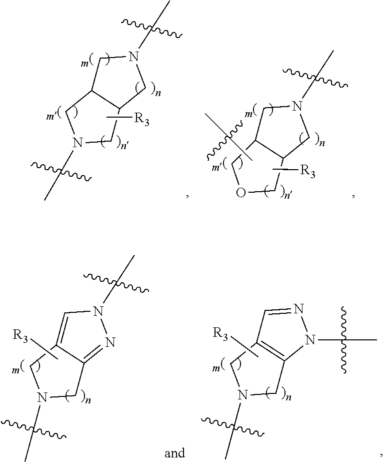
wherein the heterocyclyl contains 1, 2 or 3 nitrogen heteroatoms as a ring skeleton, and m, n, m′ and n′ are each independently 0, 1, 2 or 3;
R₃ is substituted or unsubstituted 5-6 membered heteroaryl, C1-C6 alkyl or C1-C6 heteroalkyl optionally substituted by one or more C1-C6 alkyl;
B is independently selected from the substituted or unsubstituted group consisting of 3-7 membered ring, C6-C14 aryl, 5-14 membered heteroaryl, 7-20 membered spiro or bridged ring, and the ring contains 0-3 heteroatoms selected from N, O or S; the “substituted” means being substituted by one or more substituents selected from the group consisting of deuterium, hydroxy, halogen, cyano, ester, amido, carbonyl, amino, C1-C6 alkyl, C1-C6 haloalkyl, C1-C6 thioalkyl, C1-C6 alkoxy, C1-C6 heteroalkyl, C1-C6 alkylamino, C3-C6 cycloalkyl, C3-C8 cycloalkylamino, C6-C14 aryl and 5-14 membered heteroaryl;
E is independently selected from the substituted or unsubstituted group consisting of hydrogen, C1-C6 alkyl, C1-C6 alkoxy, C3-C6 cycloalkyl, C1-C6 heteroalkyl, and 3-6 membered heterocyclyl, wherein the substituted comprises 0-5 R^{a};
each R₅ is independently selected from the substituted or unsubstituted group consisting of hydrogen, nitro, cyano, halogen, C1-C6 alkyl, C2-C6 alkenyl, C2-C6 alkynyl, C1-C6 alkoxy, C1-C6 heteroalkyl, C3-C12 cycloalkyl, C6-C14 aryl, 5-14 membered heteroaryl, C6-C14 aryloxy, C6-C14 aryl C1-C6 alkyl, 3-12 membered heterocyclyl, 3-12 membered heterocycloalkyl, —C(O)R₆, —OC(O)R₆, —C(O)OR₆, —(C1-C6 alkylene)-C(O)R₆, —SR₆, —S(O)₂R₆, —S(O)₂—N(R₆)(R₇), —(C1-C6 alkylene)-S(O)₂R₆, —(C1-C6 alkylene)-S(O)₂—N(R₆)(R₇), —N(R₆)(R₇), —C(O)—N(R₆)(R₇), —N(R₆)—C(O)R₇, —N(R₆)—C(O)OR₇, —(C1-C6 alkylene)-N(R₆)—C(O)R₇, —N(R₆)S(O)₂R₇ and —P(O)(R₆)(R₇); wherein the substituted comprises 0, 1, 2, 3, 4 or 5 R^{a}; R₆ and R₇ are each independently selected from the group consisting of C1-C6 alkyl, C2-C6 alkenyl, C2-C6 alkynyl, C1-C6 alkoxy, C1-C6 heteroalkyl, C3-C6 cycloalkyl, C6-C14 aryl, 5-14 membered heteroaryl, C6-C14 aryloxy, C6-C14 aryl C1-C6 alkyl, C3-C6 heterocycloalkyl, C1-C6 alkylamino, C3-C6 cycloalkylamino; or R₆ and R₇ together with their adjacent N atom form a substituted or unsubstituted 3-6 membered heterocyclyl; wherein the substituted comprises 0, 1, 2, 3, 4 or 5 R^{a};
A is independently selected from the group consisting of H, substituted or unsubstituted C1-C6 alkyl, substituted or unsubstituted 4-6 membered heterocyclyl, (R₁R₂N)C(═O)—; wherein the substituted comprises one or more groups selected from the group consisting of halogen, —OH, C1-C6 alkoxy, C1-C6 alkyl, amino, 5-6 membered heteroaryl, 4-6 membered heterocyclyl, C3-C6 cycloalkyl, amido, (R₁R₂N) C(═O)—, hydroxyC1-C6 alkyl, (C1-C6 alkyl) C(═O)—, C1-C6 alkoxy, oxo and (C1-C6 alkoxy) C(═O)—; R₁ and R₂ are each independently selected from H or C1-C6 alkyl, wherein the C1-C6 alkyl is optionally substituted by 1-3 fluorine;
Z₁ is selected from the group consisting of NR^{b}, S—, —C(R^{b}R^{c})— and —O—;
D is a 5-14 membered heteroaryl, wherein hydrogen on the heteroaryl is optionally substituted by one or more substituents selected from the group consisting of deuterium, hydroxy, halogen, cyano, ester, amido, oxo, amino, C1-C6 alkyl, C1-C6 alkoxy, C1-C6 heteroalkyl, C3-C6 cycloalkyl, C3-C8 cycloalkylamino, C6-C14 aryl and 5-14 membered heteroaryl; the C1-C6 alkyl, C1-C6 alkoxy, C1-C6 heteroalkyl, C3-C6 cycloalkyl, C3-C8 cycloalkylamino, C6-C14 aryl or 5-14 membered heteroaryl optionally substituted by one or more groups selected from the group consisting of halogen, cyano and hydroxyl;
f is 0, 1, 2, 3, 4, 5 or 6;
R^{a} is independently selected from the group consisting of O, C1-C6 alkyl, halogen, hydroxyl, C1-C6 heteroalkyl, C1-C6 alkoxy, C3-C14 cycloalkyl, 3-14 membered heterocycloalkyl and cyano;
R^{b} and R^{c} are independently selected from the group consisting of H, C1-C6 alkyl, halogen, hydroxyl, C1-C6 heteroalkyl, C1-C6 alkoxy, C3-C14 cycloalkyl, 3-14 membered heterocycloalkyl and cyano;
with the proviso that
when 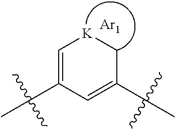
is 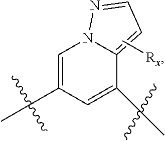
Ar₂ is a 5-6 membered heteroaryl, and Ar₂ connects with ring Q₂ or 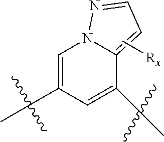
through N; wherein Rₓ is selected from the group consisting of H, CN, halogen, methyl, ethyl and cyclopropyl; and wherein the hydrogen on Ar₂ is optionally substituted by CR^{a}.

2. The compound, or the pharmaceutically acceptable salt, stereoisomer, solvate or prodrug thereof of claim 1, has the structure represented by formula (I), formula (II), formula (III) formula (IV) formula (V) or formula (VI), 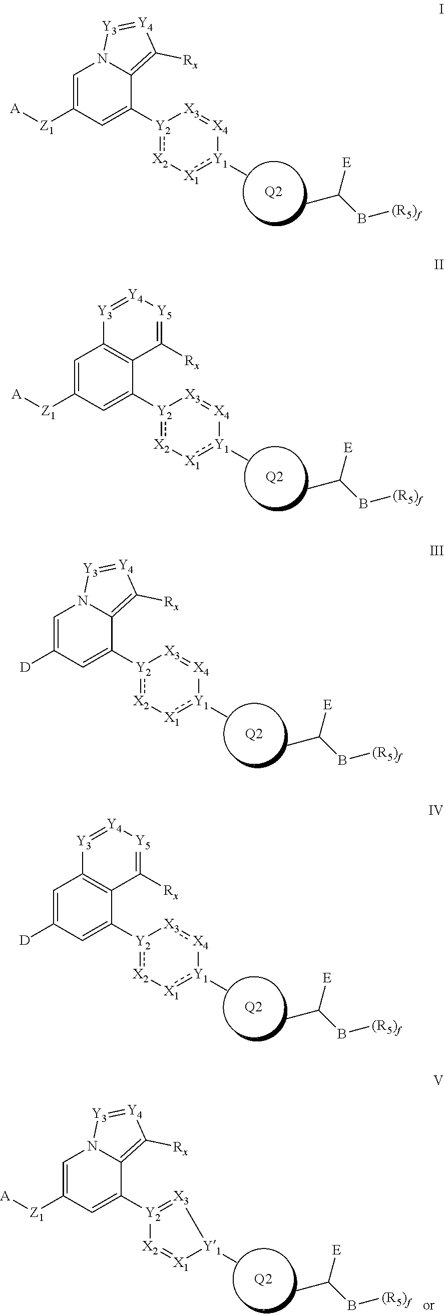 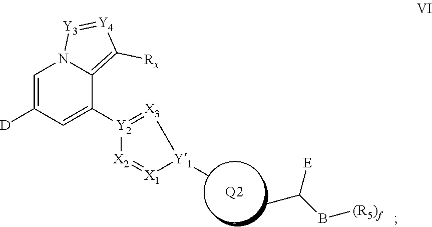
wherein 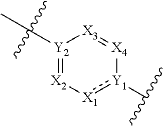
is a six-membered heteroaryl; 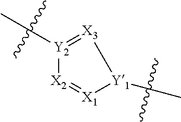
is a five-membered heteroaryl;
X₁, X₂, X₃ and X₄ are each independently selected from the group consisting of CH, N or CR^{a}, and 0, 1 or 2 of X₁, X₂, X₃ and X₄ are N;
Y′₁ is N;
Y₁ is C or N;
Y₃ and Y₅ are each independently CH, N, or CR^{a};
Y₂ is N or C;
Y₄ is CH, N, or CR^{a};
Rₓ is independently selected from the group consisting of H, CN, halogen, methyl, ethyl and cyclopropyl;
with the proviso that in formula I and formula III, when Y₃ is N and Y₄ is CH or N, Y₁ or Y₂ are N.

3. The compound, or the pharmaceutically acceptable salt, stereoisomer, solvate or prodrug thereof of claim 1, wherein Ar₁ is the substituted or unsubstituted group consisting of 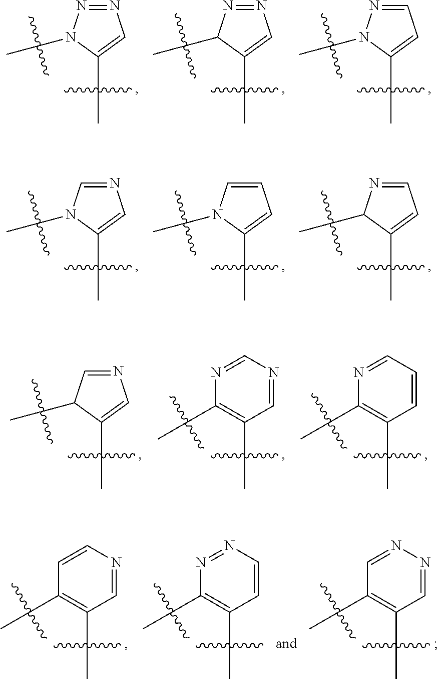
wherein the substituted comprises one or more groups selected from the group consisting of H, CN, halogen, methyl, ethyl and cyclopropyl.

4. The compound, or the pharmaceutically acceptable salt, stereoisomer, solvate or prodrug thereof of claim 1,
wherein the 5- or 6-membered heteroaryl in Ar₂ is 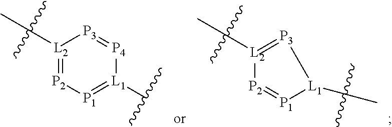
wherein P₁, P₂, P₃ and P₄ are each independently selected from N or CH wherein 0, 1 or 2 of P₁, P₂, P₃ and P₄ are N; and
L₁ and L₂ are each independently selected from N or C.

5. The compound, or the pharmaceutically acceptable salt, stereoisomer, solvate or prodrug thereof of claim 1, wherein Q₂ is a saturated 5-6 membered monocyclic heterocyclyl containing one or two nitrogen ring heteroatoms, and the hydrogen on Q₂ is optionally substituted by one or more substituents selected from the group consisting of deuterium, hydroxy, halogen, cyano, ester, amido, oxo, carbonyl, amino, C1-C6 alkyl, C1-C6 haloalkyl, C1-C6 thioalkyl, C1-C6 alkoxy, C1-C6 heteroalkyl, C1-C6 alkylamino, C3-C6 cycloalkyl, C3-C8 cycloalkylamino, C6-C14 aryl and 5-14 membered heteroaryl.

6. The compound, or the pharmaceutically acceptable salt, stereoisomer, solvate or prodrug thereof of claim 2, wherein Y₃ is N.

7. The compound, or the pharmaceutically acceptable salt, stereoisomer, solvate or prodrug thereof of claim 1, wherein B is a 5-6 membered heteroaryl and the hydrogen on B is optionally substituted by one or more substituents selected from the group consisting of deuterium, hydroxy, halogen, cyano, ester, amido, carbonyl, amino, C1-C6 alkyl, C1-C6 haloalkyl, C1-C6 thioalkyl, C1-C6 alkoxy, C1-C6 heteroalkyl, C1-C6 alkylamino, C3-C6 cycloalkyl, C3-C8 cycloalkylamino, C6-C14 aryl and 5-14 membered heteroaryl.

8. The compound, or the pharmaceutically acceptable salt, stereoisomer, solvate or prodrug thereof of claim 1, having the structure represented by formula (VII), formula (VIII) or formula (IX), 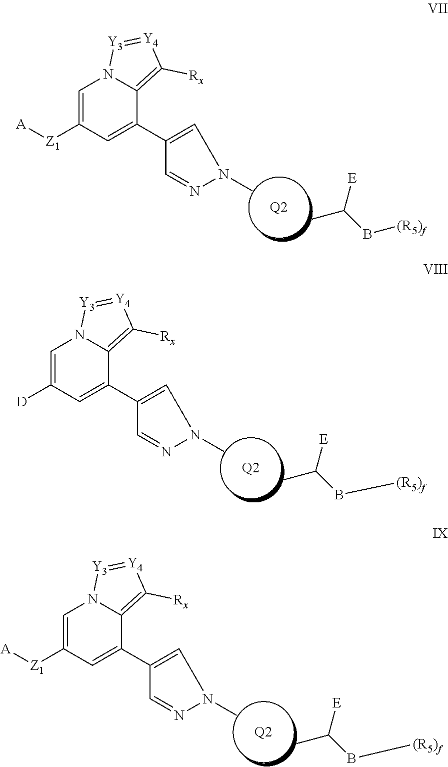
wherein
Y₃ and Y₄ are each independently CH, N, or CR^{a}.

9. The compound, or the pharmaceutically acceptable salt, stereoisomer, solvate or prodrug thereof of claim 1, having the structure represented by formula (XI) or formula (XIII), 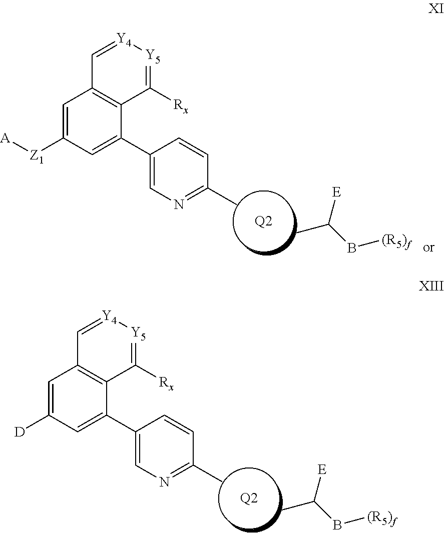
wherein Y₄ and Y₅ are each independently CH, N or CR^{a}.

10. The compound, or the pharmaceutically acceptable salt, stereoisomer, solvate or prodrug thereof of claim 1, having the structure represented by formula (XIV): 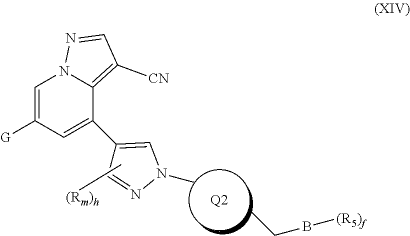
wherein,
each Rₘ is independently selected from C1-C6 alkyl, halogen, hydroxyl, oxo (═O), C1-C6 heteroalkyl, C1-C6 alkoxy, C3-C14 cycloalkyl, 3-14 membered heterocycloalkyl or cyano; an
h is 0, 1 or 2.

11. The compound, or the pharmaceutically acceptable salt, stereoisomer, solvate or prodrug thereof of claim 1, wherein the compound is selected from the group consisting of:

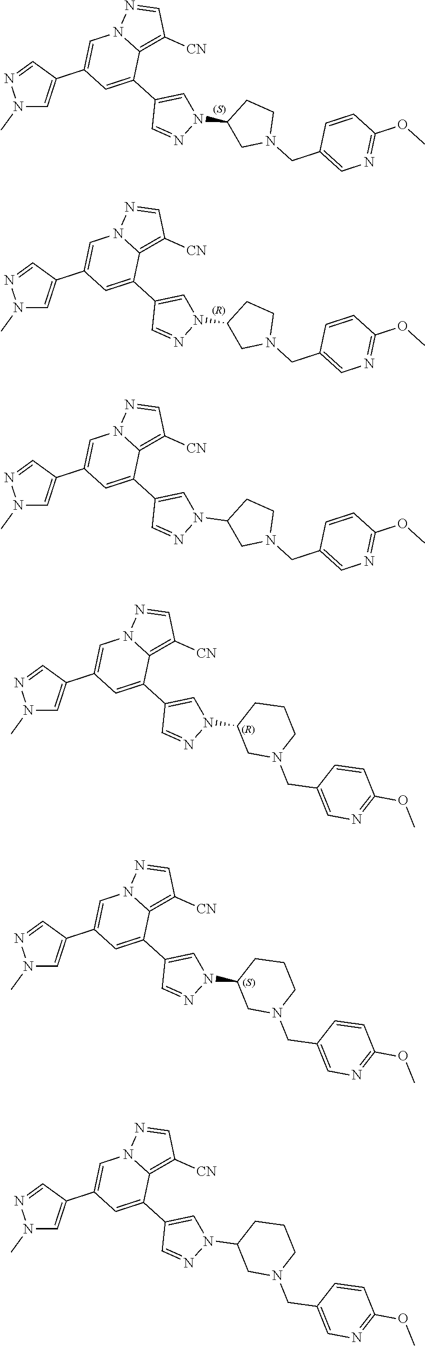

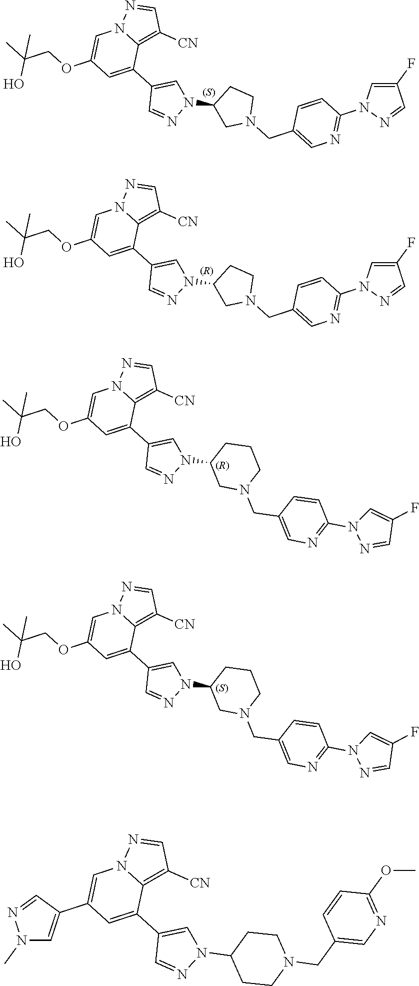

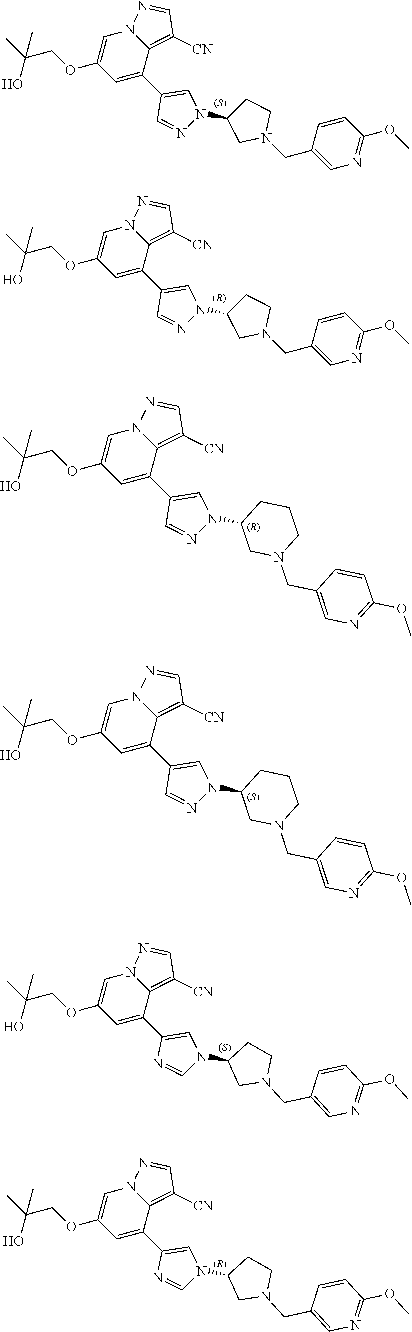

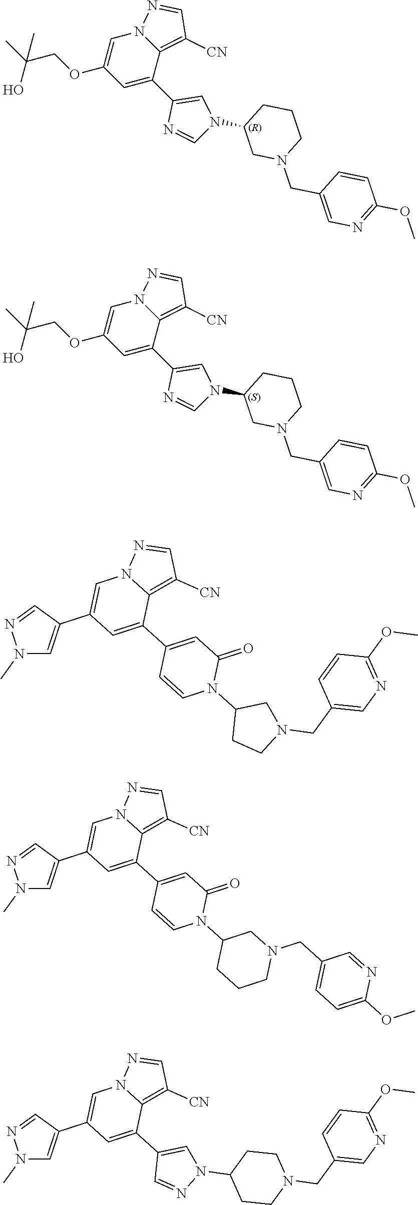

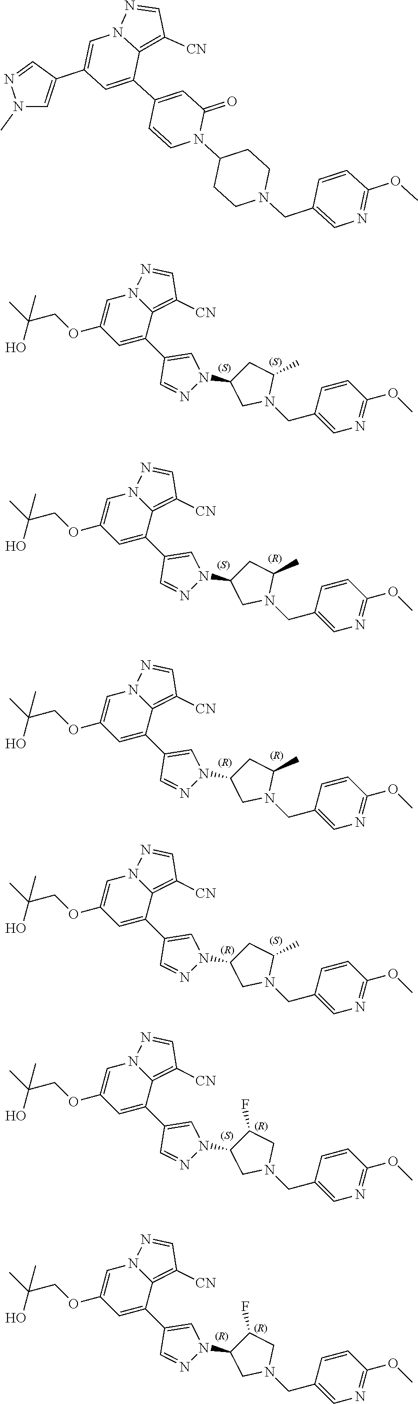

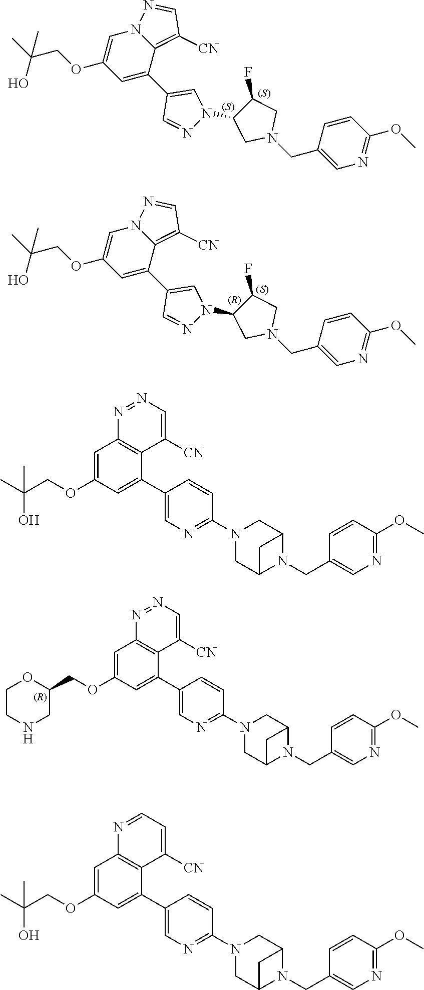

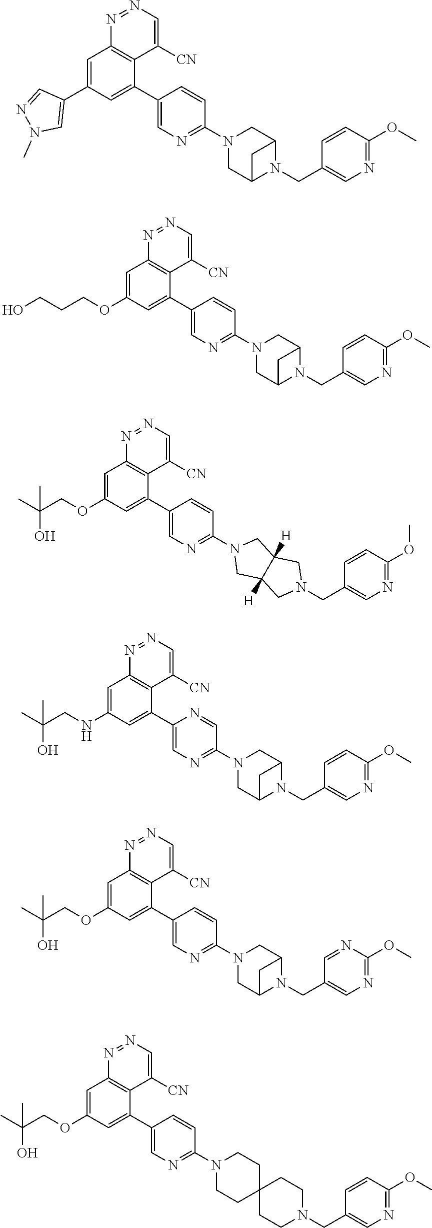

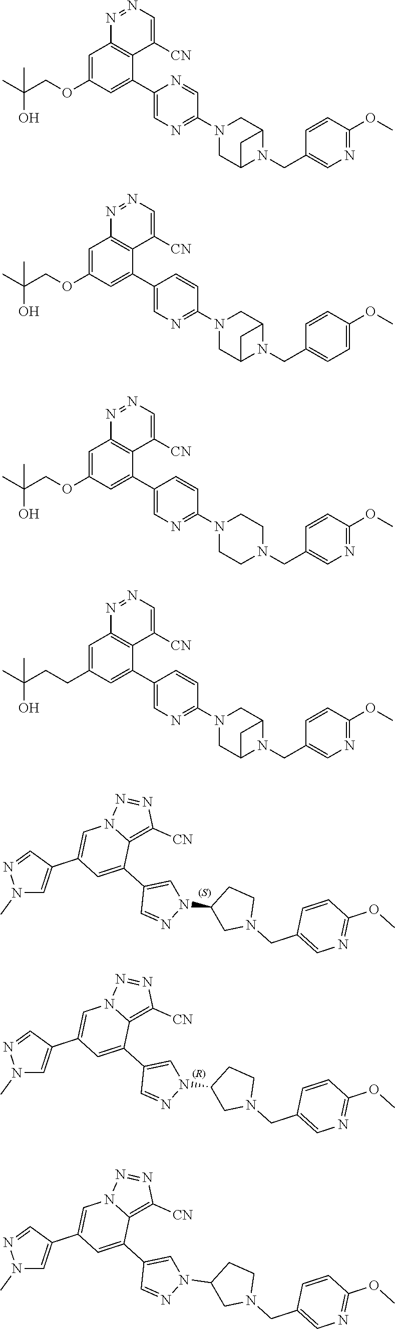

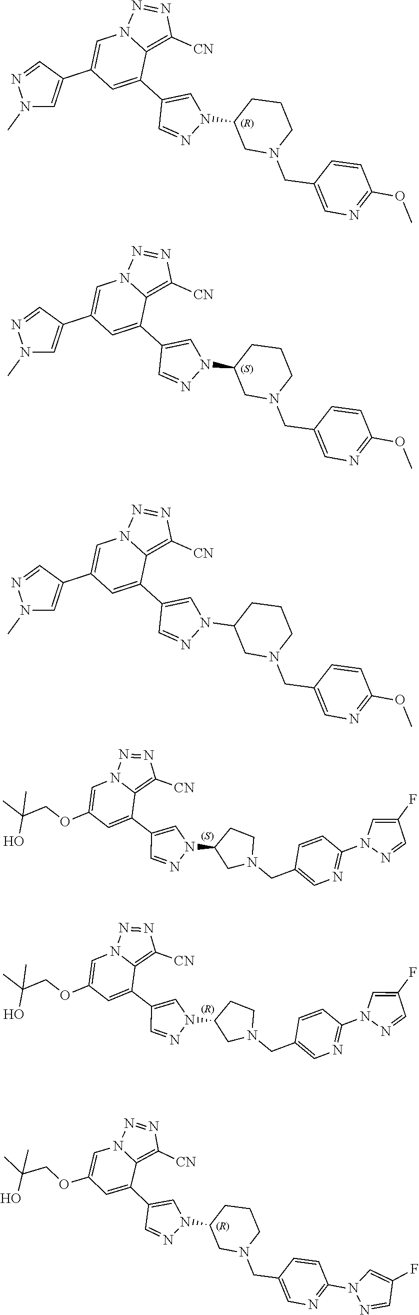

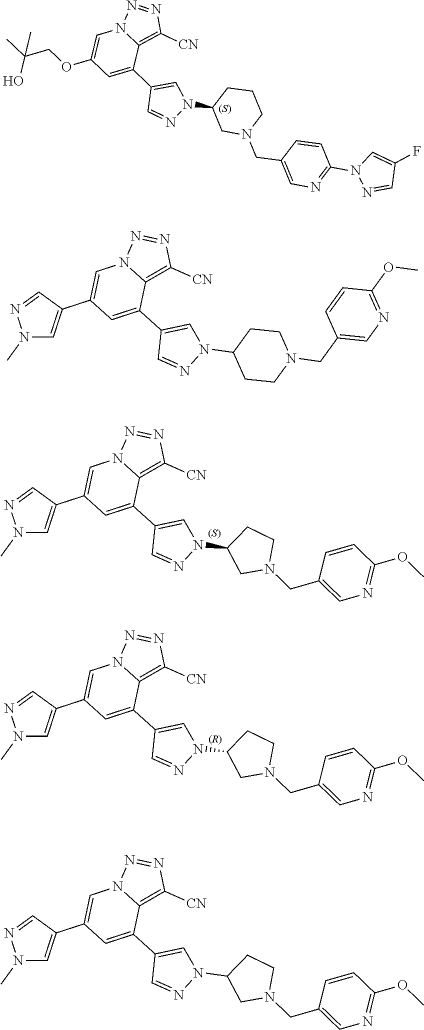

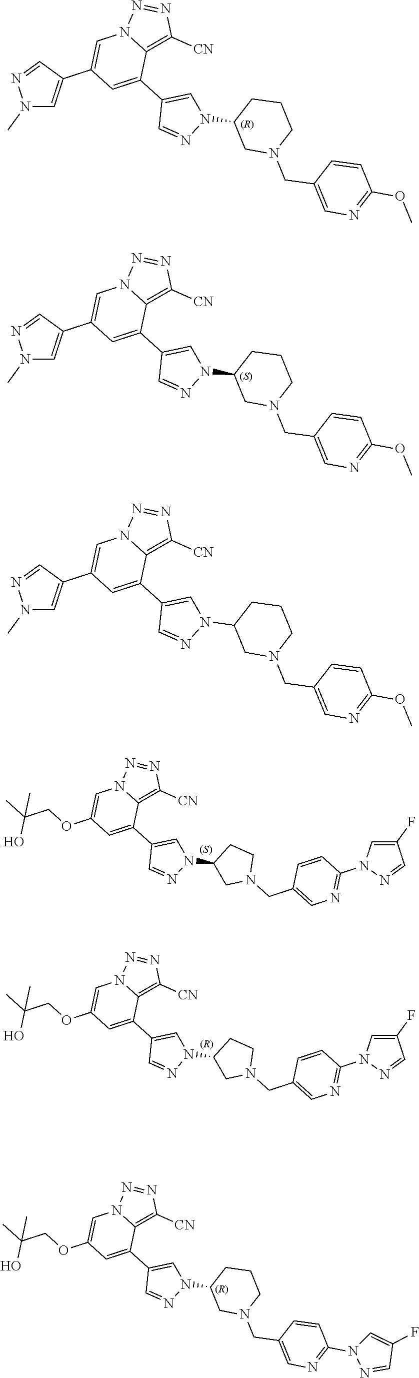

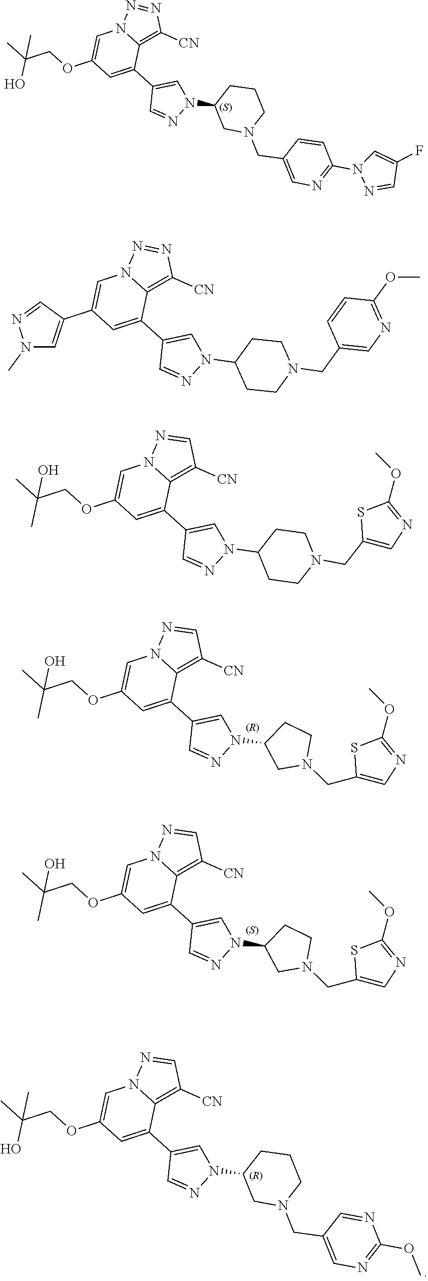

12. A pharmaceutical composition comprising the compound, or the pharmaceutically acceptable salt, stereoisomer, solvate or prodrug thereof of claim 1; and a pharmaceutically acceptable carrier.

13. A method for inhibiting RET kinase activity comprising the steps of contacting a therapeutically effective amount of the compound, or the pharmaceutically acceptable salt, stereoisomer, solvate or prodrug thereof of claim 1 with a cell or a subject, thereby inhibiting the RET kinase activity.

14. A method for treating RET-related cancer comprising administering a therapeutically effective amount of the compound, or the pharmaceutically acceptable salt, stereoisomer, solvate or prodrug thereof of claim 1 to a subject in need thereof.

15. The method of claim 14, wherein the RET-related cancer is selected from the group consisting of lung cancer, papillary thyroid carcinoma, medullary thyroid carcinoma, differentiated thyroid carcinoma, recurrent thyroid carcinoma, refractory differentiated thyroid carcinoma, multiple 2A or 2B endocrine tumors (MEN2A or MEN2B respectively), pheochromocytoma, parathyroid hyperplasia, breast cancer, colorectal cancer, papillary renal cell carcinoma, gastrointestinal mucosal ganglioneuroma and cervical cancer.

16. The compound of claim 1, wherein the hydrogen on Q₂ is substituted by one or more substituents selected from the group consisting of deuterium, hydroxy, halogen, cyano, ester, amido, carbonyl, oxo (═O), amino, C1-C6 alkyl, C1-C6 haloalkyl, C1-C6 thioalkyl, C1-C6 alkoxy, C1-C6 heteroalkyl, C1-C6 alkylamino, C3-C6 cycloalkyl, C3-C8 cycloalkylamino, C6-C14 aryl and 5-14 membered heteroaryl.

17. The method of claim 14, wherein the subject is a mammal.

18. The method of claim 14, wherein the subject is resistant to other cancer treatment.

19. The composition of claim 12, further comprising a PD-1 inhibitor, a PD-L1 inhibitor, a CD20 antibody, a CD47 antibody, an ALK inhibitor, a PI3K inhibitor, a BTK inhibitor, a VEGFR inhibitor, a HDAC inhibitor, a CDK inhibitor, a MEK inhibitor, an Akt inhibitor, a MTOR inhibitor, a SHP2 inhibitor, an IGF-1R inhibitor, or a combination thereof.

20. The composition of claim 12, further comprising nivolumab, pembrolizumab, JS-001, SHR-120, BGB-A317, IBI-308, GLS-010, GB-226, STW204, HX008, HLX10, BAT1306, AK105, LZM 009, devaluzumab, Atezolizumab, CS1001, KN035, HLX20, SHR-1316, BGB-A333, JS003, CS1003, KL-A167, F520, GR1405, MSB2311, rituximab, obinutuzumab, ofatumumab, tositumomab, titumomab, Hu5F9-G4, CC-90002, TTI-621, TTI-622, OSE-172, SRF-231, ALX-148, NI-1701, SHR-1603, IBI188, IMM01, ceritinib, alectinib, brigatinib, loratinib, okatinib, idelaris, Dactolisib, Taselisib, Buparlisib, ibrutinib, Tirabrutinib, Acalabrutinib, afatinib, gefitinib, erlotinib, lapatinib, dacomitinib, ectinib, Tinib, canetinib, sorafenib, pazopanib, revatinib, carbotinib, sunitinib, donafinib, Givinostat, Droxinostat, entinostat, daxistat, tycdinaline, Pabocinil, Ribocinil, Abemaciclib, Lerociclib, Metinib, trametinib, PD0325901, U0126, AS-703026, PD184352, MK-2206, Ipatasertib, Capivasertib, Afuresertib, Uprosertib, Vistusertib, RMC-4630, JAB-3068, TNO155, Ceritinib, okatinib, linsitinib, BMS-754807, GSK1838705A, or a combination thereof.
